(19) United States
(12) Patent Application Publication
(10) Pub. No.: US 2013/0274270 A1
(43) Pub. Date: Oct. 17, 2013

(54) POLYMORPHS OF 3-CHLORO-4[(2R)-2-(4-CHLOROPHENYL)-4-[(1R)-1-(4-CYANOPHENYL)ETHYL]-1-PIPERAZINYL]-BENZONITRILE, PHARMACEUTICAL COMPOSITIONS AND METHOD OF USE COMPRISING SAID POLYMORPHS, AND A PROCESS FOR PREPARING THEM

(71) Applicants: Monika Brink, Gau-Algesheim (DE); Hans-Peter Niedermann, Bubenheim (DE); Marcus Knell, Hochheim (DE); Tao Feng, Shanghai (CN); Scott T. Trzaska, Summit, NJ (US); Arthur J. Cooper, Concord, OH (US); Shaileshkumar Ramanial Desai, Wilmington, DE (US); Vinayak Keshav Gore, Wilmington, DE (US)

(72) Inventors: Monika Brink, Gau-Algesheim (DE); Hans-Peter Niedermann, Bubenheim (DE); Marcus Knell, Hochheim (DE); Tao Feng, Shanghai (CN); Scott T. Trzaska, Summit, NJ (US); Arthur J. Cooper, Concord, OH (US); Shaileshkumar Ramanial Desai, Wilmington, DE (US); Vinayak Keshav Gore, Wilmington, DE (US)

(21) Appl. No.: 13/995,389

(22) PCT Filed: Dec. 21, 2011
(86) PCT No.: PCT/IB2011/003110

§ 371 Date: Jun. 18, 2013

Related U.S. Application Data
(60) Provisional application No. 61/426,592, filed on Dec. 23, 2010.

(30) Foreign Application Priority Data
Feb. 18, 2011 (EP) 11155049.7

(51) Int. Cl. C07D 295/155; A61K 45/06; A61K 31/495

(52) U.S. Cl. CPC C07D 295/155; A61K 31/495; A61K 45/06
USPC 514/255.03; 544/392; 558/418

(57) ABSTRACT

Polymorphs of 3-chloro-4[(2R)-2-(4-chlorophenyl)-4-[(1R)-1-(4-cyanophenyl)ethyl]-1-piperazinyl]-benzonitrile are disclosed, as well as pharmaceutical compositions comprising said polymorphs, methods of using the polymorphs, and a process for preparing them.

### FIELD OF THE INVENTION

The invention relates to crystalline polymorphs of the cannabinoid receptor 1 (CB1) antagonists 3-chloro-4[(2R)-2-(4-chlorophenyl)-4-[(1R)-1-(4-cyanophenyl)ethyl]-1-piperazinyl]-benzonitrile, pharmaceutical compositions containing said polymorphs, and methods and formulations comprising said polymorphs useful in treating obesity. The invention also relates to a process for preparing 3-chloro-4[(2R)-2-(4-chlorophenyl)-4-[(1R)-1-(4-cyanophenyl)ethyl]-1-piperazinyl]-benzonitrile and related compounds.

### BACKGROUND OF THE INVENTION

3-Chloro-4[(2R)-2-(4-chlorophenyl)-4-[(1R)-1-(4-cyanophenyl)ethyl]-1-piperazinyl]-benzonitrile, having the Formula A,

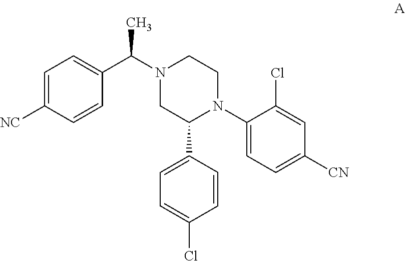 [SMILES: C[C@H](c1ccc(C#N)cc1)N1CCN(c2ccc(C#N)cc2Cl)[C@H](c2ccc(Cl)cc2)C1]

and related compounds are disclosed in U.S. Pat. No. 7,700,597, issued Apr. 20, 2010; the compound (Example 392a in the patent) is identified as a CB1 antagonist indicated to be useful in the treatment of conditions such as metabolic syndrome (e.g. obesity), heptic lipidosis, neuroinflammatory disorders and cognitive disorders in animals. In addition, the antagonists could also be used as partitioning agents in animals.

Processes disclosed in U.S. Pat. No. 7,700,597 for making the compounds disclosed therein are not suitable for large-scale manufacture. The presently claimed process provides a safer, more convergent and more economical method of preparing the compounds.

### SUMMARY OF THE INVENTION

The invention provides crystalline polymorphs of the compound of Formula A wherein, said polymorph is selected from the group consisting of:
Form I that exhibits a powder x-ray diffraction pattern substantially the same as the pattern shown in FIG. 1;
Form II that exhibits a powder x-ray diffraction pattern substantially the same as the pattern shown in FIG. 2; and
Form III that exhibits a powder x-ray diffraction pattern substantially the same as the pattern shown in FIG. 3.
   In another embodiment, the invention provides the crystalline polymorph Form I of the compound of Formula A that exhibits a powder x-ray diffraction pattern substantially the same as the pattern shown in FIG. 1.

The invention further provides a crystalline polymorph Form I of the compound of Formula A that exhibits a powder x-ray diffraction pattern having characteristic peak locations of 13.4, 20.2, 23.0 and 24.1 degrees 2θ.

In another embodiment, the crystalline polymorph Form I exhibits a powder x-ray diffraction pattern having characteristic peak locations of 13.4, 17.9, 20.2, 20.6, 23.0, 24.1, 25.9 and 27.6 degrees 2θ.

In another embodiment, the crystalline polymorph Form I exhibits a powder x-ray diffraction pattern having characteristic peak locations of 13.4, 17.9, 18.6, 20.2, 20.6, 21.2, 23.0, 24.1, 25.5, 25.9, 27.6 and 30.5 degrees 2θ.
In another embodiment, the invention provides the crystalline polymorph Form I that exhibits a melt substantially the same as shown in the differential scanning calorimetry scan of FIG. 4.
In another embodiment, the invention provides the crystalline polymorph Form I that exhibits a melt substantially the same as shown in the differential scanning calorimetry scan having a characteristic melt of 127.7° C.
In another embodiment, the invention provides the crystalline polymorph Form II that exhibits a powder x-ray diffraction pattern substantially the same as the pattern shown in FIG. 2.

The invention further provides a crystalline polymorph Form II of the compound of Formula A that exhibits a powder x-ray diffraction pattern having characteristic peak locations of 12.9, 15.8, 18.5 and 24.2 degrees 2θ.

In another embodiment, the crystalline polymorph Form II exhibits a powder x-ray diffraction pattern having characteristic peak locations of 9.5, 12.9, 15.8, 18.5, 21.2, 22.2, 22.7 and 24.2 degrees 2θ.

In another embodiment, the crystalline polymorph Form II exhibits a powder x-ray diffraction pattern having characteristic peak locations of 9.5, 12.9, 15.8, 18.5, 21.2, 22.2, 22.7, 24.2, 25.6, 26.4, 27.8 and 28.2 degrees 2θ.
In another embodiment, the invention provides the crystalline polymorph Form II that exhibits a melt substantially the same as shown in the differential scanning calorimetry scan of FIG. 5.
In another embodiment, the invention provides the crystalline polymorph Form II that exhibits a melt substantially the same as shown in the differential scanning calorimetry scan having a characteristic melt of 113.0° C.
In another embodiment, the invention provides the crystalline polymorph Form III that exhibits a powder x-ray diffraction pattern substantially the same as the pattern shown in FIG. 3.

The invention further provides a crystalline polymorph Form III of the compound of Formula A that exhibits a powder x-ray diffraction pattern having characteristic peak locations of 17.9, 21.9, 24.5 and 26.8 degrees 2θ.

In another embodiment, the crystalline polymorph Form III exhibits a powder x-ray diffraction pattern having characteristic peak locations of 7.3, 13.4, 17.9, 19.2, 21.9, 24.5, 25.2 and 26.8 degrees 2θ.

In another embodiment, the crystalline polymorph Form III exhibits a powder x-ray diffraction pattern having characteristic peak locations of 7.3, 12.9, 13.4, 17.9, 19.2, 20.1, 21.1, 21.6, 21.9, 24.5, 25.2 and 26.8 degrees 2θ.

In another embodiment, the invention provides the crystalline polymorph Form III that exhibits a melt substantially the same as shown in the differential scanning calorimetry scan of FIG. 6.

In another embodiment, the invention provides the crystalline polymorph Form III that exhibits a melt substantially the same as shown in the differential scanning calorimetry scan having a characteristic melt of 154.8° C.

The invention further provides a pharmaceutical composition comprising a crystalline polymorph selected from the group consisting of Form I, Form II, and Form III, or a mixture thereof, and at least one excipient or carrier.

The invention further provides a purified form of the polymorph Form I.

The invention further provides a purified form of the polymorph Form II.

The invention further provides a purified form of the polymorph Form III.

The invention further provides a method of treating an obesity related disease in a patient in need of such treatment, comprising administering to said patient an effective amount of at least one polymorph of compound A.

The invention further provides a method of treating obesity in a patient in need of such treatment comprising administering to said patient an effective amount of at least one polymorph of compound A.

The invention further provides a method of treating obesity related disease in a patient in need of such treatment, wherein the obesity related disease is as disclosed in U.S. Pat. No. 7,700597. The invention further provides a method of treating obesity related disease such as diabetes and hepatic lipidosis. The invention further provides a class of compounds which can be combined with other molecules to achieve even better efficacy then when used alone. A few examples of molecules which could be combined with a CB-1 antagonist include but are not limited to NYP5 inhibitors, Histamine-3 antagonist, lipase inhibitors and lipid absorption inhibitors such as MTP inhibitors and DGAT-1 inhibitors.

Another aspect of the invention relates to the preparation of compounds of Formula I

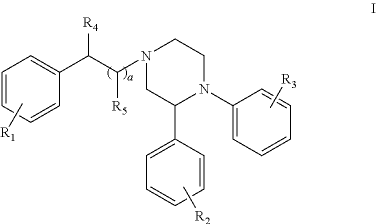

wherein R₁ is one or two substituents independently selected from the group consisting of H, halo, alkyl and cyano;

R₂ is one or two substituents independently selected from the group consisting of H, halo, alkyl and cyano;

R₃ is one or two substituents independently selected from the group consisting of H, halo, alkyl and cyano; and

R₄ is H or alkyl, and a is 0; or R₅ is H, alkyl, hydroxyalkyl or alkoxyalkyl, and a is 1; comprising:

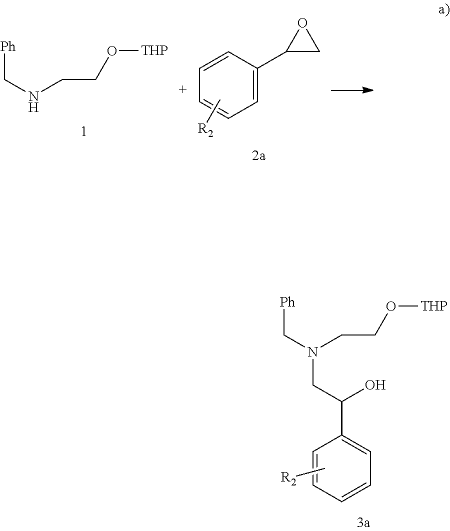

reacting the protected amino ethanol 1 and 4-chloro-styreneoxide (2a), wherein Ph is phenyl and THP is tetrahydropyranyl, to obtain compound 3a;

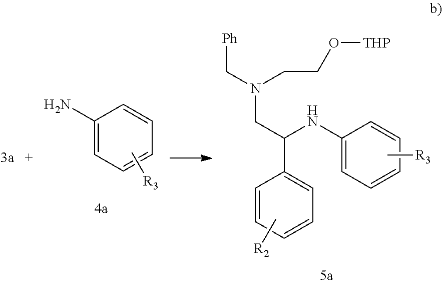

reacting compound 3a and 4-amino-3-chlorobenzonitrile (4a) in the presence of methanesulfonyl chloride and a base to obtain compound 5a;

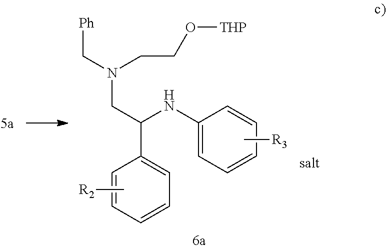

removing the THP protecting group from compound 5a and converting the product to a salt (6a);

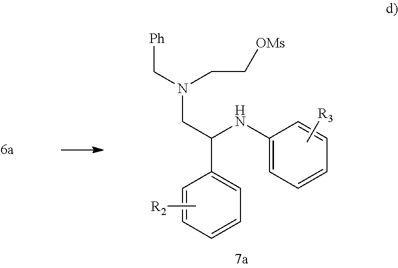

treating compound 6a with methanesulfonyl (Ms) chloride and a base to obtain compound 7a;

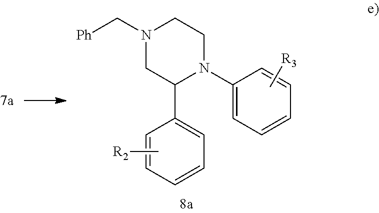

cyclizing compound 7a by treating with sodium hydroxide to obtain 8a;

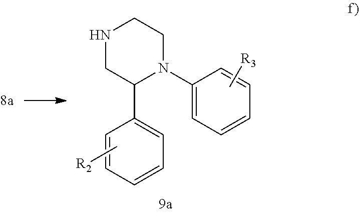

removing the benzyl protecting group from compound 8a to obtain 9a; and

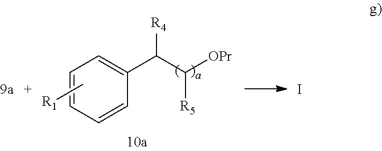

reacting compound 9a with compound 10a, wherein Pr is a protecting group, in the presence of potassium carbonate.

Further, the present invention is a process for preparing the compound of the Formula II, which is an embodiment of Formula I wherein R₁ is cyano, R₂ is chloro, R₃ is chloro and cyano, a is 0 and R₄ is methyl, and which is the racemate of Formula A

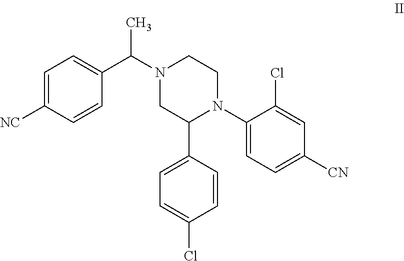 [SMILES: CC(c1ccc(C#N)cc1)N1CCN(c2ccc(C#N)cc2Cl)C(c2ccc(Cl)cc2)C1]

comprising:

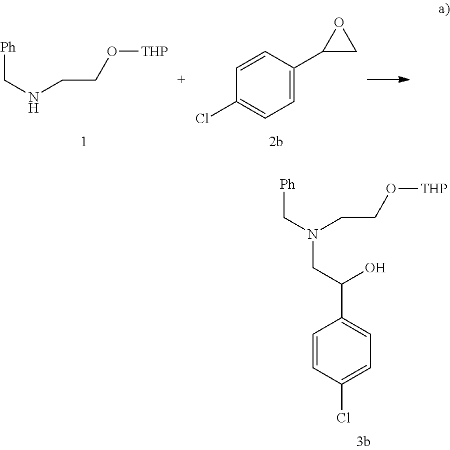

reacting the protected amino ethanol 1b and 4-chloro-styreneoxide (2b), wherein Ph is phenyl and THP is tetrahydropyranyl, to obtain compound 3b;

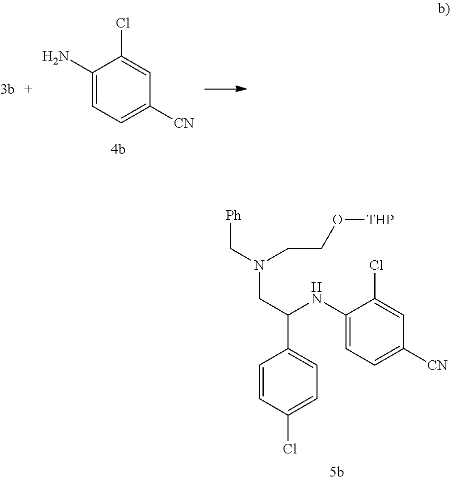

reacting compound 3b and 4-amino-3-chlorobenzonitrile (4b) in the presence of methanesulfonyl chloride and a base to obtain compound 5b;

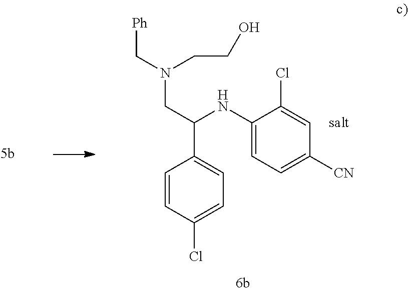 [SMILES: N#Cc1ccc(NC(CN(CCO)Cc2ccccc2)c2ccc(Cl)cc2)c(Cl)c1]

removing the THP protecting group from compound 5b and converting the product to a salt (6b);

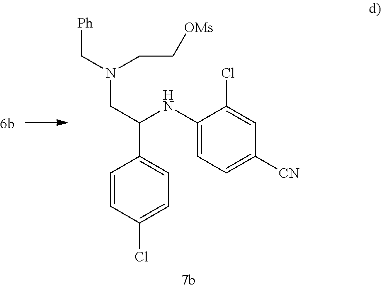 [SMILES: CS(=O)(=O)OCCN(Cc1ccccc1)CC(Nc1ccc(C#N)cc1Cl)c1ccc(Cl)cc1]

treating compound 6b with methanesulfonyl (Ms) chloride and a base to obtain compound 7b;

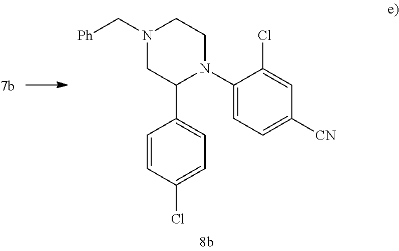 [SMILES: N#Cc1ccc(N2CCN(Cc3ccccc3)CC2c2ccc(Cl)cc2)c(Cl)c1]

cyclizing compound 7b by treating with sodium hydroxide to obtain 8b;

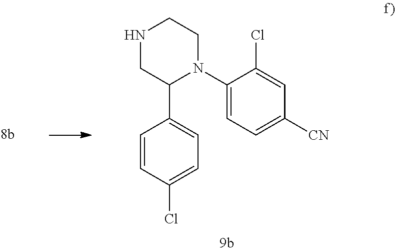 [SMILES: N#Cc1ccc(N2CCNCC2c2ccc(Cl)cc2)c(Cl)c1]

removing the benzyl protecting group from compound 8b; and

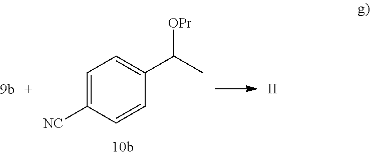

reacting compound 9b with compound 10b, wherein Pr is a protecting group, in the presence of potassium carbonate.

In another embodiment, the invention relates to the preparation of the compound of Formula A according the process described above for Formula II, but by using the enantiomeric intermediates as described below,

comprising:

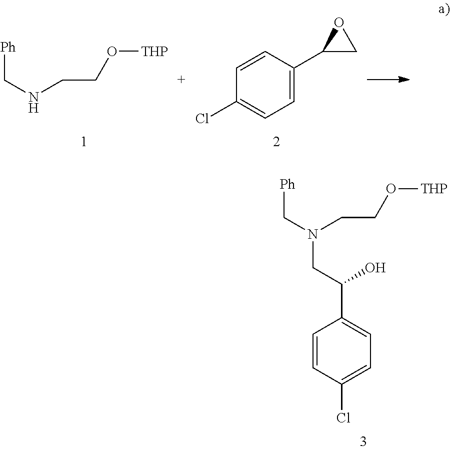

reacting the protected amino ethanol 1 and 4-chloro-styreneoxide (2), wherein Ph is phenyl and THP is tetrahydropyranyl, to obtain compound 3;

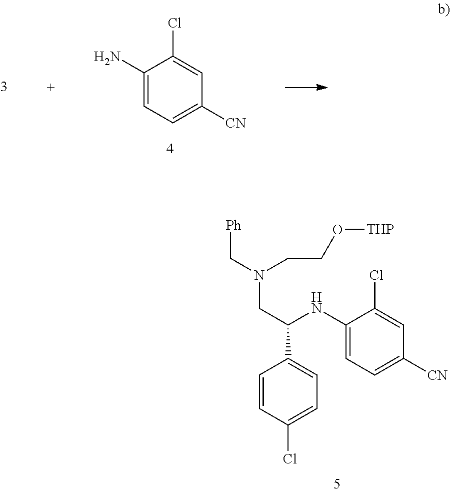

reacting compound 3 and 4-amino-3-chlorobenzonitrile (4) in the presence of methanesulfonyl chloride and a base to obtain compound 5;

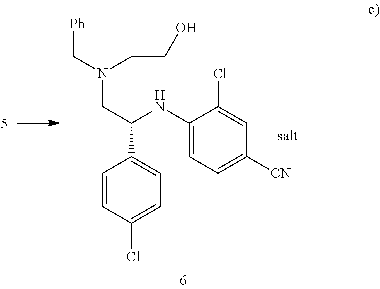 [SMILES: N#Cc1ccc(N[C@@H](CN(CCO)Cc2ccccc2)c2ccc(Cl)cc2)c(Cl)c1]

removing the THP protecting group from compound 5 and converting the product to a salt (6);

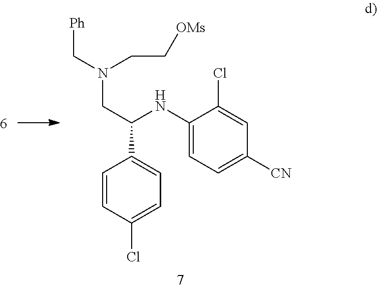 [SMILES: CS(=O)(=O)OCCN(Cc1ccccc1)C[C@H](Nc1ccc(C#N)cc1Cl)c1ccc(Cl)cc1]

treating compound 6 with methanesulfonyl (Ms) chloride and a base to obtain compound 7;

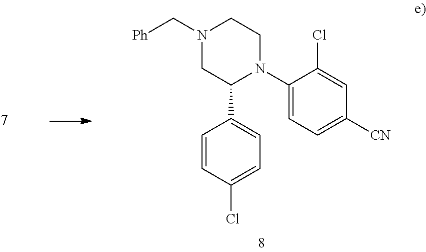 [SMILES: N#Cc1ccc(N2CCN(Cc3ccccc3)C[C@H]2c2ccc(Cl)cc2)c(Cl)c1]

cyclizing compound 7 by treating with sodium hydroxide to obtain 8;

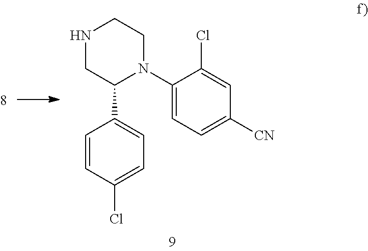 [SMILES: N#Cc1ccc(N2CCNC[C@H]2c2ccc(Cl)cc2)c(Cl)c1]

removing the benzyl protecting group from compound 8 to obtain 9; and

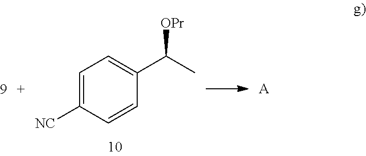

reacting compound 9 with compound 10, wherein Pr is a protecting group, in the presence of potassium carbonate.

The present invention also relates to the following novel intermediates:

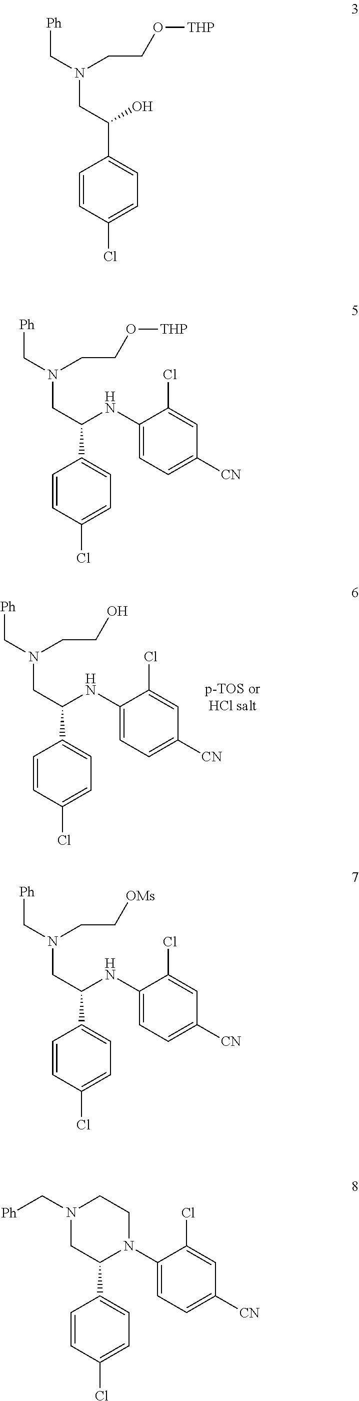

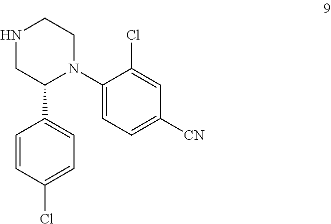 [SMILES: N#Cc1ccc(N2CCNC[C@H]2c2ccc(Cl)cc2)c(Cl)c1]

### BRIEF DESCRIPTION OF THE DRAWINGS

FIG. 1 is a graph of a powder x-ray diffraction (PXRD) pattern of Form I of Compound A, generated using an X-ray diffractometer. The graph plots the intensity of the peaks as defined by counts per second versus the diffraction angle 2θ in degrees.

FIG. 2 is a graph of a PXRD pattern of Form II of Compound A. The graph was generated using an X-ray diffractometer. The graph plots the intensity of the peaks as defined by counts per second versus the diffraction angle 2θ in degrees.

FIG. 3 is a graph of a PXRD pattern of Form III of Compound A, generated using an X-ray diffractometer. The graph plots the intensity of the peaks as defined by counts per second versus the diffraction angle 2θ in degrees.

FIG. 4 is a differential scanning calorimetry scan of Form I of Compound A. The graph plots heat flow (W/g) versus temperature (° C.).

FIG. 5 is a differential scanning calorimetry scan of Form II of Compound A. The graph plots heat flow (W/g) versus temperature (° C.).

FIG. 6 is a differential scanning calorimetry scan of Form III of Compound A. The graph plots heat flow (W/g) versus temperature (° C.).

### DETAILED DESCRIPTION OF THE INVENTION

### Definitions

In the above summary and throughout the specification, the following terms are defined as follows:
“halo” refers to chloro, bromo. fluoro or iodo;
“alkyl” means a C₁-C₆ alkyl group, branched or straight;
“hydroxyl alkyl” means a C₁-C₆ alkyl group having a hydroxyl substituent;
“alkoxyalkyl” means a C₁-C₆ alkyl-O— group joined to a C₁-C₆ alkyl group;
“polymorph” means a crystalline form of a substance that is distinct from another crystalline form but that shares the same chemical formula;
“inventive polymorph” means any of the three crystalline polymorphs Forms I-III of Formula A and is not limited to a single polymorph but can include more than one form;
“patient” includes both human and other mammals;
“excipient” means an essentially inert substance used as a diluent or to give form or consistency to a formulation;
   “effective” or “therapeutically effective” is meant to describe a polymorph of a compound or a composition of the present invention effective as a chemokine receptor ligand and thus producing the desired therapeutic, ameliorative, inhibitory or preventative effect; and
   “effective amount” or “therapeutically effective amount” is meant to describe an amount of polymorph or a composition of the present invention effective as a chemokine receptor ligand and thus producing the desired therapeutic, ameliorative, inhibitory or preventative effect.

### Description of Polymorphs

The present invention relates to Form I, II and II polymorphs as described above; polymorphs are significant because the physical attributes of a material such as solubility, stability, and melting point are impacted by the crystal form. It is necessary to reproducibly manufacture a material in the same crystal form to minimize variability in performance as well as ensure consistent quality. Identification and development of the thermodynamically most stable crystal form of a material is important because this form will have the lowest susceptibility to conversion to another form during manufacturing, storage, and processing.

The presently claimed polymorphs were identified by solvent-mediated variable-temperature slurry experiments conducted using the amorphous free base form of the molecule. Solids were isolated from these experiments and a competition slurry experiment was designed to determine which polymorphic form was the most thermodynamically stable. During these experiments, Form I was identified as a stable form and possessed acceptable physical properties. Form I is converted into Form II. Form III, was identified during the development of the claimed process for preparing the compounds of Formula A. Competition slurry experiments confirmed that Form III was the most stable form.

### Experimental Procedures For Determining Polymorphs

The polymorph Form I is prepared from amorphous Compound A by the process comprising:
   a) mixing amorphous Compound A at room temperature in a first mixture of an alcohol and water to form a second mixture;
   b) adding water dropwise until the second mixture becomes hazy;
   c) adding the organic solvent dropwise until the second mixture becomes clear, and
   d) allowing the second mixture to stand at room temperature until Form I crystals precipitate.
Preferably, the alcohol is methanol or ethanol.
The polymorph Form II is prepared from Form I by mixing the Form I material with an organic solvent as a slurry at room temperature until Form II crystals precipitate.
Preferably, the organic solvent is methylene chloride or acetone.
The polymorph Form III is prepared from amorphous Compound A by a process comprising:
   a. mixing amorphous Compound A at elevated temperature with a first quantity of an organic solvent to form a mixture;
   b. adding water portion-wise until precipitate is detected;
   c. adding a second quantity of the organic solvent;
   d. heating the mixture to about 70° C.; and
   e. allowing the mixture to stand at room temperature until Form III crystals precipitate.
Preferably, the organic solvent is n-propanol.
Preferably, the ratio of the first quantity to the second quantity is about 2:1.
Preferably, the first mixture comprises n-propanol and water in a ratio of about 1.1:1.

The polymorphs were characterized using the following procedures.

All powder X-ray diffraction (PXRD) data was collected using a Rigaku Miniflex instrument. Samples were analyzed on silicon zero background holders from 2 to 40 degrees two theta with a step size of 0.02 degrees two theta and a scan time of 0.6 seconds per step. Differential scanning calorimetry (DSC) data was collected using a Q100 DSC from TA Instruments. Samples were sealed in hermetic aluminum pans with two pinholes in the lid. All were analyzed under nitrogen purge. Vapor sorption analysis was performed using a SGA-100 from VTI Scientific Instruments. Thermogravimetric analysis (TGA) data was collected on a Q500 TGA from TA Instruments. Samples were analyzed in platinum pans under a nitrogen purge.

Solvent-mediated polymorphic transformation experiments were conducted by combining approximately 100 mg of starting material with 1 milliliter of solvent in a 4 mL amber glass vial. A polytetrafluoroethylene coated magnetic stir bar was added to each vial. For suspensions, the vials were sealed with polytetrafluoroethylene-lined caps and stirred using magnetic stir plates at 4° C., room temperature, or 50° C. for 14 days. For solutions, the vials were covered with aluminum foil and the solutions were slowly evaporated for 14 days. Solids were collected and analyzed for changes in crystal form.

Competition slurry experiments were conducted by combining approximately 100 milligrams of starting material with 1 milliliter of solvent in a 4 milliliter amber glass vial. A polytetrafluoroethylene coated magnetic stir bar was added to each vial. The slurry sample was stirred for one day and checked to confirm solids were still present. The sample was then seeded with forms identified during the polymorph screening and continued stirring at room temperature for 9 days. Solids were collected and analyzed to determine their crystal form.

Change in crystal form as a function of moisture was evaluated by a SGA-100 vapor sorption instrument from VTI Scientific Instruments. Approximately 10 to 15 mg of material was exposed to a relative humidity range from 5% to 95% at 25° C. for 2 cycles.

Change in crystal form as a function of milling was conducted by grinding 79.2 milligrams of material in a stainless steel liner with a stainless steel ball pestle using a high speed mixer and amalgamator. The material was ground for 5 seconds at 3800 rpm.

Change in crystal form as a function of pressure was evaluated by pressing 86.0 mg of material in a 13 millimeter die at approximately 4000 pounds for approximately 30 seconds with a carver press.

Change in crystal form as a function of heating was evaluated by analyzing samples of the material using the DSC at heating rates of 2, 10, and 20° C. per minute.

Nine different acids including sulfuric, phosphoric, tartaric, citric, malic, fumaric, ketoglutaric, xinafoic, and malonic acid were explored as part of the salt screening. The free base of Compound A was combined with sulfuric acid in a 0.5 to 1 molar ratio and all other acids in a 1:1 molar ratio in isopropanol, tetrahydrofuran, and toluene. All salt-forming reactions that produced solutions were concentrated by slow evaporation of the solvent to produce crystalline salts. All salt-forming reactions that produced suspensions were stirred in an attempt to produce crystalline salts.

### Results of Polymorph Experiments

For this work, both slurry and evaporative crystallization was performed. Slurry experiments are utilized to identify the thermodynamically stable form while evaporative crystallizations favor the identification of metastable forms.

All slurry experiments except those in water produced a polymorph termed Form I regardless of the starting form, water activity, or temperature indicating Form I was the most thermodynamically stable form. In all slurry experiments in water, the starting form was recovered suggesting that the rate of polymorphic conversion in water is slower than the time scale of the experiments.

The evaporative crystallization experiments from solutions produced another polymorph, termed Form II, and two suspected solvates or hydrates. Competition slurries were prepared with Form I, Form II and the suspected solvates or hydrates to confirm they were metastable with respect to Form 1. The competition slurries were prepared by dispersing Form II in isopropanol, a water/methanol mixture at a water activity of 0.5, and water. Solids collected from the competition slurry experiments in isopropanol and the water/methanol mixture were consistent with Form I. Solids collected from the competition slurry experiment in water stayed as Form II and can be attributed to the slow conversion kinetics previously noted in water during the polymorph screening.

The results for solvent-mediated polymorphic transformation using Form I as the starting material are summarized in Table 1. The results for the solvent-mediated polymorphic transformations using Form II as the starting material are summarized in Table 2.

TABLE 1
Solvent-Mediated Polymorphic Transformation Results using Form I

| Experiment | Solvent | Temperature/State | PXRD Result |
|---|---|---|---|
| 1 | Ethyl Acetate | RT/Solution | Suspected Solvate or |
| | | | Hydrate |
| 2 | Isopropanol | RT/Suspension | Form 1 |
| 3 | Toluene | RT/Solution | Suspected Solvate or |
| | | | Hydrate |
| 4 | Acetone | RT/Solution | Suspected Solvate or |
| | | | Hydrate |
| 5 | Dichloromethane | RT/Solution | Form 2 |
| 6 | Acetonitrile | RT/Solution | Form 2 |
| 7 | Tetrahydrofuran | RT/Solution | Suspected Solvate or |
| | | | Hydrate |
| 8 | Water | RT/Suspension | Form 1 |
| 9 | Water/Methanol | RT/Suspension | Form 1 |
| 10 | Water/Methanol | 4° C./Suspension | Form 1 |
| 11 | Water/Methanol | 50° C./Suspension | Form 1 |
| 12 | Toluene | 4° C./Solution | Suspected Solvate or |
| | | | Hydrate |
| 13 | Toluene | 50° C./Solution | Amorphous |

TABLE 2
Solvent-Mediated Polymorphic Transformation Results using Form II

| Experiment | Solvent | Temperature/State | PXRD Result |
|---|---|---|---|
| 1 | Ethyl Acetate | RT/Solution | Suspected Solvate or |
| | | | Hydrate |
| 2 | Isopropanol | RT/Suspension | Form I |
| 3 | Toluene | RT/Solution | Suspected Solvate or |
| | | | Hydrate |
| 4 | Acetone | RT/Solution | Suspected Solvate or |
| | | | Hydrate |
| 5 | Dichloromethane | RT/Solution | Form II |
| 6 | Acetonitrile | RT/Solution | Form II |
| 7 | Tetrahydrofuran | RT/Solution | Suspected Solvate or |
| | | | Hydrate |
| 8 | Water | RT/Suspension | Form II |
| 9 | Water/Methanol | RT/Suspension | Form I |
| 10 | Toluene | 4° C./Solution | Suspected Solvate or |
| | | | Hydrate |
| 11 | Water/Methanol | 4° C./Suspension | Form I |
| 12 | Toluene | 50° C./Solution | Suspected Solvate or |
| | | | Hydrate |
| 13 | Water/Methanol | 50° C./Suspension | Form I |

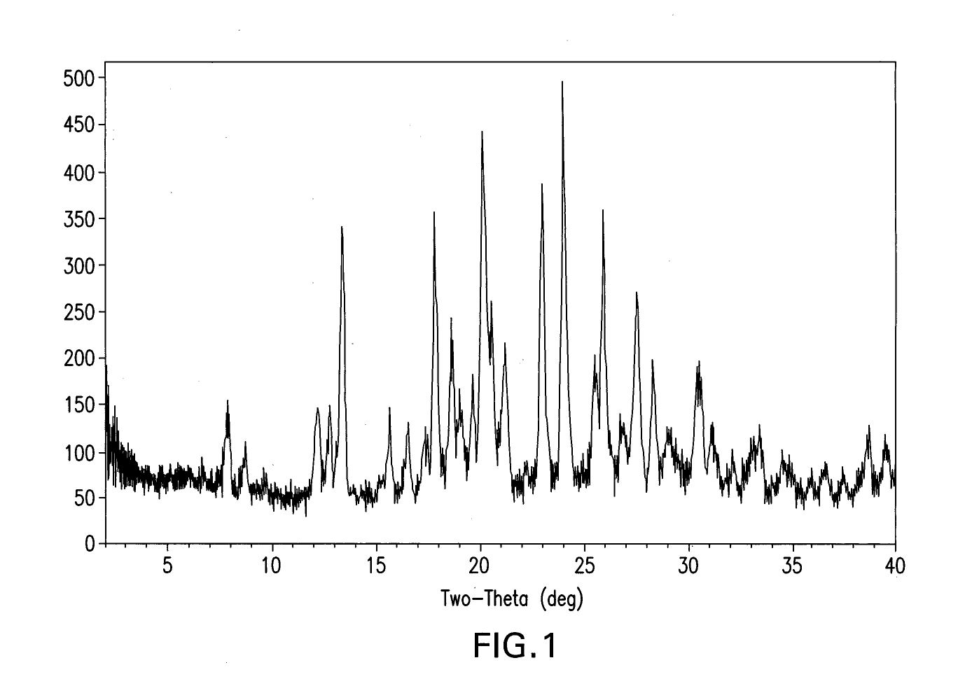

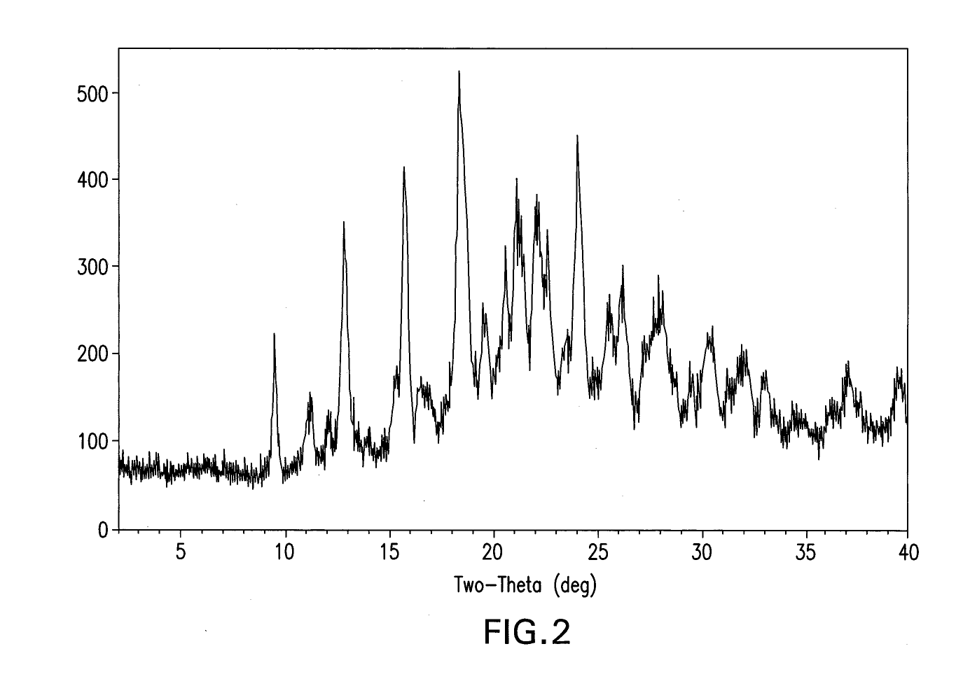

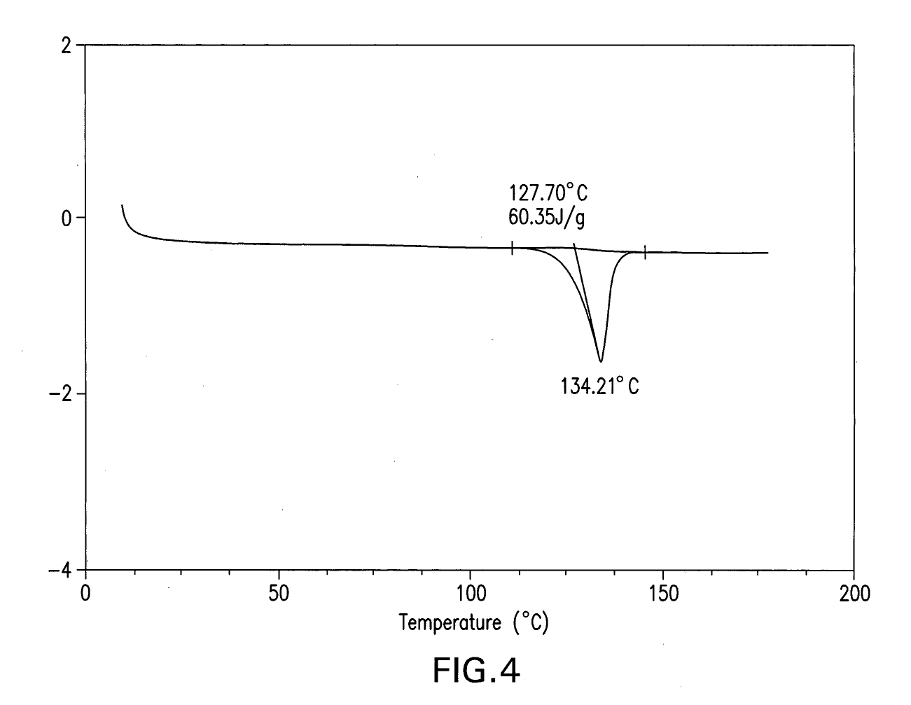

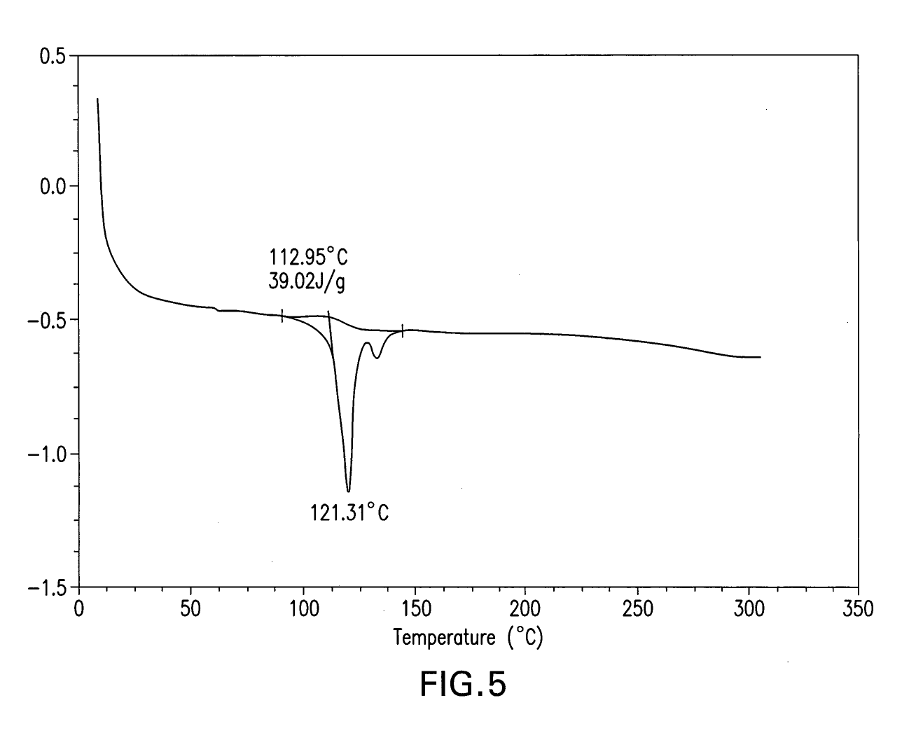

Forms I and II were analyzed using PXRD (FIGS. 1 and 2), DSC (FIGS. 4 and 5), and TGA. Both Form I and Form II had several sharp, unique peaks in the PXRD, indicating that both forms were crystalline.

Form I had a melting point of 127.7° C., a weight loss of 0.6% at 204° C., and decomposed above 295° C. The enthalpy of fusion for Form I was 60.4 J/g. Form II had a melting point of 113.0° C., a weight loss of 0.6% at 226° C., and decomposed at 310° C. The enthalpy of fusion for Form II was 39.0 J/g.

The DSC data collected suggested that Form I had a higher melting point and larger heat of fusion that Form II. Based upon the heat of fusion rule, Form I and Form II were determined to be monotropically related and Form I the more thermodynamically stable.

Vapor sorption data collected on Form I demonstrated that it absorbed only 0.07% moisture by weight between 5% and 95% relative humidity. The material was analyzed by PXRD before and after the vapor sorption analysis, confirming no polymorphic form change.

The milling experiments demonstrated no significant change in the crystal form after milling.

The high pressure experiments demonstrated no significant change in the crystal form of Form I after pressing in a Carver press.

Form I was heated at 2° C., 10° C., and 20° C. per minute using the DSC instrument. The scans had no unusual endotherms or exotherms. Therefore, there was no change in the crystal form as a function of heating rate.

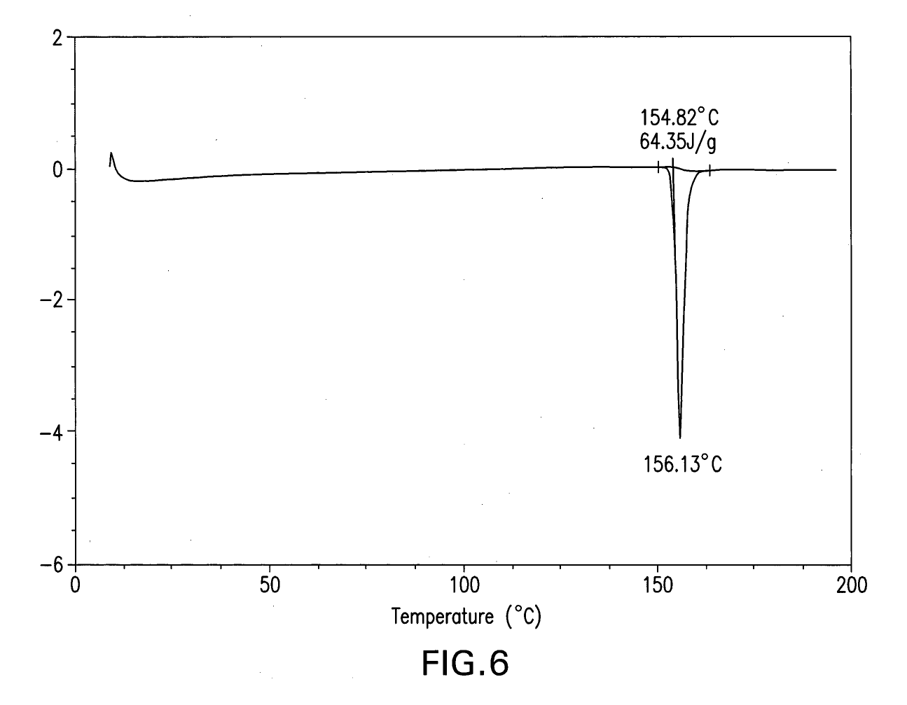

During the development of the claimed process, an additional form was found, termed Form III. Characterization was performed utilizing DSC (FIG. 6) and TGA. Form III had a melting point of 154.8° C., a weight loss of 0.07% at 228° C., and decomposed at 312° C. Its enthalpy of fusion was 64.4J/g.

A variable-temperature competition slurry was repeated using Forms I and III by suspending approximately 100 mg of each form in 2 milliliters of isopropanol at 4° C., room temperature, 40° C., and 50° C. The slurries were allowed to stir for 11 days and checked by PXRD. The results showed that Form III was formed under all conditions. Afterwards, the solutions were seeded with Form I and stirred for 1 day. The PXRD results showed that the material remained as Form III.

To further characterize Form III, temperature-solubility profiles were constructed for both Form I and III. Different amounts of each form were suspended in 1 milliliter of isopropanol to prepare solutions of varying concentrations. The experiments were conducted on Crystal 16 to generate a temperature-solubility profile. The suspensions were slowly heated from 20° C. to 75° C. at 0.1° C./min and then cooled to 0° C. at a rate of 0.1° C. /min. The stirring rate was 700 rpm during the entire experiment. Form III was shown to be less soluble than Form I at temperatures greater than 29.4° C., thereby more stable above this temperature and the most thermodynamically stable anhydrous crystalline form of the molecule.

The solids resulting from the salt screening procedure were isolated for PXRD analysis. The results for the screening are summarized in Table 3.

TABLE 3
Summary of Salt Screening Results for Compound A

| Experiment | Acid | Method | Result |
|---|---|---|---|
| 1 | Sulfuric acid/0.5 eq | Evaporation | Amorphous |
| 2 | Phosphoric acid | Evaporation | Amorphous |
| 3 | Tartaric acid | Stirring | Free base |
| 4 | Citric acid | Stirring | Amorphous |
| 5 | Malic acid | Stirring | Free base |
| 6 | Fumaric acid | Stirring | Free base |
| 7 | Ketoglutaric acid | Stirring | Free base |
| 8 | Xinafoic acid | Stirring | Crystalline xinafoate |
| 9 | Malonic acid | Stirring | Free base |

The xinafoate salt was found to be crystalline with a weight loss of 0.2% at 146° C. and 27.2% starting from 161° C. and ending at 236° C. It has a melt at 163.5° C., with a large weight loss suggesting that the salt disproportionates or decomposes with melting.

### Polymorph Purity

Preferably, the crystalline polymorphs Forms I-III of Compound A are substantially free of chemical impurities (e.g., by-products generated during the preparation of the polymorphs) and of other polymorphic crystalline forms. “Substantially free” of chemical impurities for the purposes of this invention means less than or equal to about 5% w/w of chemical impurities, preferably, less than or equal to about 3% w/w of chemical impurities, more preferably, less than or equal to about 2% w/w of chemical impurities, and even more preferably, less than or equal to about 1% w/w of chemical impurities. The term “purified” or “in purified form” for a polymorph refers to the physical state of said polymorph after being obtained from a purification process or processes described herein or well known to the skilled artisan, in sufficient purity to be characterizable by standard analytical techniques described herein or well known to the skilled artisan. Purified forms of the crystalline polymorph Forms I-IV of Compound A are substantially free of chemical impurities.

### Pharmaceutical Compositions

For preparing pharmaceutical compositions from one or more of the polymorphs described by this invention, inert, pharmaceutically acceptable carriers can be either solid or liquid. Solid form preparations include powders, tablets, dispersible granules, capsules, cachets and suppositories. The powders and tablets may be comprised of from about 5 to about 95 percent active ingredient. Suitable solid carriers are known in the art, e.g., magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18^{th} Edition, (1990), Mack Publishing Co., Easton, Pa.

Liquid form preparations include solutions, suspensions and emulsions. As an example may be mentioned water or water-propylene glycol solutions for parenteral injection or addition of sweeteners and opacifiers for oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also included are solid form preparations that are intended to be converted, shortly before use, to liquid form preparations for either oral or parenteral administration. Such liquid forms include solutions, suspensions and emulsions.

An inventive polymorphs may also be deliverable transdermally. The transdermal composition can take the form of creams, lotions, aerosols and/or emulsions and can be included in a transdermal patch of the matrix or reservoir type as are conventional in the art for this purpose.

In some embodiments, an inventive polymorph or combination of polymorphs is administered orally.

In some embodiments, the pharmaceutical preparation is in a unit dosage form. In such form, the preparation is subdivided into suitably sized unit doses containing appropriate quantities of the active component, e.g., an effective amount to achieve the desired purpose.

### Dosages

The quantity of active compound in a unit dose of preparation is defined in U.S. Pat. No. 7,700,597. In an embodiment, it may be varied or adjusted from about 0.01 mg to about 1000 mg, preferably from about 0.01 mg to about 750 mg, more preferably from about 0.01 mg to about 500 mg, and most preferably from about 0.01 mg to about 250 mg, and most preferably from about 0.01 mg to about 100 mg according to the particular application.

The actual dosage employed may be varied depending upon the requirements of the patient and the severity of the condition being treated. Determination of the proper dosage regimen for a particular situation is within the skill of the art. For convenience, the total dosage may be divided and administered in portions during the day as required.

The amount and frequency of administration of the compounds of the invention and/or the pharmaceutically acceptable salts thereof will be regulated according to the judgment of the attending clinician considering such factors as age, condition and size of the patient as well as severity of the symptoms being treated. A typical recommended daily dosage regimen for oral administration can range from about 0.04 mg/day to about 4000 mg/day, in one to four divided doses.

### Co-Formulations

In some embodiments of the treatment of obesity, at least one of the polymorphs disclosed herein is administered in combination with an additional therapeutic agent as disclosed in U.S. Pat. No. 7,700,597.

Methods for the safe and effective administration of most of these therapeutic agents are known to those skilled in the art. In addition, their administration is described in the standard literature. For example, the administration of many of the therapeutic agents is described in the “Physicians' Desk Reference” (PDR), e.g., 2002 edition (Medical Economics Company, Montvale, N.J. 07645-1742, USA); and “Veterinary Pharamaceuticals and Biologicals” (The Veterinarian's PDR), e.g. 1999/2000 edition (Medical Economics Company, Montvale, N.J. 07645-1742, USA), the disclosures of which are incorporated herein by reference thereto.

Another embodiment of the invention is directed to a method treating obesity, comprising administering to a patient in need thereof, concurrently or sequentially, a therapeutically effective amount of (a) at least one of the polymorphs disclosed herein, and (b) an additional agent as disclosed in U.S. Pat. No. 7,700,597.

The inventive polymorph, and therapeutic agent may be administered concurrently (e.g., simultaneously, essentially simultaneously or within the same treatment protocol) or sequentially, depending upon the nature of the proliferative disease, the condition of the patient, and the actual choice of therapeutic agent to be administered in conjunction (i.e., within a single treatment protocol) with the inventive polymorph.

Also, in general, the inventive polymorph and the therapeutic agent do not have to be administered in the same pharmaceutical composition, and may, because of different physical and chemical characteristics, have to be administered by different routes. For example, the inventive polymorph may be administered orally to generate and maintain good blood levels thereof, while the therapeutic agent may be administered intravenously. The determination of the mode of administration and the advisability of administration, where possible, in the same pharmaceutical composition, is well within the knowledge of the skilled clinician. The initial administration can be made according to established protocols known in the art, and then, based upon the observed effects, the dosage, modes of administration and times of administration can be modified by the skilled clinician.

The particular choice of an inventive polymorph, and therapeutic agent will depend upon the diagnosis of the attending physicians and their judgment of the condition of the patient based on body weight and various body score criteria available form pet food industries such as Purina body score and the appropriate treatment protocol.

If the inventive polymorph, and the therapeutic agent are not administered simultaneously or essentially simultaneously, then the initial order of administration of the inventive polymorph, and the therapeutic agent, may not be important. Thus, the inventive polymorph may be administered first, followed by the administration of the therapeutic agent; or the therapeutic agent may be administered first, followed by the administration of the inventive polymorph. This alternate administration may be repeated during a single treatment protocol. The determination of the order of administration, and the number of repetitions of administration of each therapeutic agent during a treatment protocol, is well within the knowledge of the skilled physician after evaluation of the disease being treated and the condition of the patient.

In accordance with experience and knowledge, the practicing physician can modify each protocol for the administration of a component (therapeutic agent—i.e., the inventive polymorph or therapeutic agent) of the treatment according to the individual patient's needs, as the treatment proceeds.

Other than as shown in the operating examples or as otherwise indicated, all numbers used in the specification and claims expressing quantities of ingredients, reaction conditions, and so forth, are understood as being modified in all instances by the term “about.” The above description is not intended to detail all modifications and variations of the invention. It will be appreciated by those skilled in the art that changes can be made to the embodiments described above without departing from the inventive concept. It is understood, therefore, that the invention is not limited to the particular embodiments described above, but is intended to cover modifications that are within the spirit and scope of the invention, as defined by the language of the following claims.

### Description of the Process For Preparing Compounds of Formulae I, II And A

The present process described above in the Summary of the Invention has several advantages over the process for preparing the compounds of Formula I, II and A disclosed in U.S. Pat. No. 7,700,597. Compared to the published process, the present process uses sodium hydroxide for ring closure instead of the hazardous reagent sodium hydride. Only two intermediates, 6 and 9 (or the corresponding 6a and 9a or 6b and 9b), need to be isolated, compared to six isolated intermediates in the published procedure, thus simplifying the procedure and saving time. Purification of the intermediates by column chromatography has been eliminated. The present process also generally eliminates the use of chlorinated solvents by replacing dichloromethane (DCM), preferably with 2-methyl-tetrahydrofuran (2-Me-THF). The use of sodium sulfate for drying is generally eliminated, as well as the need for solvent removal by distillation to dryness.

A preferred embodiment of the present process comprises the process for preparing the compound of Formula A.

In another embodiment, the compound of Formula A is prepared under the following conditions:
a) reacting the protected amino ethanol 1 with 4-chloro-styreneoxide (2) at about 125° C. to obtain compound 3;
b) reacting compound 3 and 4-amino-3-chlorobenzonitrile (4) in 2-methyl-tetrahydrofuran the presence of methanesulfonyl chloride and triethylamine to obtain compound 5;
c) removing the THP protecting group from compound 5 and converting the product to the p-toluenesulfonic (p-TOS) or hydrochloride salt (6);
d) treating compound 6 with methanesulfonyl (Ms) chloride and triethylamine to obtain compound 7;
e) cyclizing compound 7 by refluxing with sodium hydroxide to obtain 8;
f) removing the benzyl protecting group from compound 8 by treating with 1-chloroethyl chloroformate to obtain 9; and
g) reacting compound 9 with compound 10, wherein Pr is a mesyl group, in the presence of potassium carbonate.

In another embodiment, the compound of Formula I is prepared under the following conditions:
a) reacting the protected amino ethanol 1 with 4-chloro-styreneoxide (2a) at about 125° C. to obtain compound 3a;
b) reacting compound 3a and 4-amino-3-chlorobenzonitrile (4a) in 2-methyl-tetrahydrofuran the presence of methanesulfonyl chloride and triethylamine to obtain compound 5a;
c) removing the THP protecting group from compound 5a and converting the product to the p-toluenesulfonic (p-TOS), hydrochloride or napadisylate salt (6a);
d) treating compound 6a with methanesulfonyl (Ms) chloride and triethylamine to obtain compound 7a;
e) cyclizing compound 7a by refluxing with sodium hydroxide to obtain 8a;
f) removing the benzyl protecting group from compound 8a by treating with 1-chloroethyl chloroformate to obtain 9a; and
g) reacting compound 9a with compound 10a, wherein Pr is a mesyl group, in the presence of potassium carbonate.

In another embodiment, the compound of Formula II is prepared under the following conditions:
a) reacting the protected amino ethanol 1 with 4-chloro-styreneoxide (2b) at about 125° C. to obtain compound 3b;
b) reacting compound 3b and 4-amino-3-chlorobenzonitrile (4b) in 2-methyl-tetrahydrofuran the presence of methanesulfonyl chloride and triethylamine to obtain compound 5b;
c) removing the THP protecting group from compound 5b and converting the product to the p-toluenesulfonic (p-TOS), hydrochloride or napadisylate salt (6b);
d) treating compound 6b with methanesulfonyl (Ms) chloride and triethylamine to obtain compound 7b;
e) cyclizing compound 7b by refluxing with sodium hydroxide to obtain 8b;
f) removing the benzyl protecting group from compound 8b by treating with 1-chloroethyl chloroformate; and
g) reacting compound 9b with compound 10b, wherein Pr is a mesyl group, in the presence of potassium carbonate.

In another embodiment, the invention relates to a process for preparing compound A comprising:

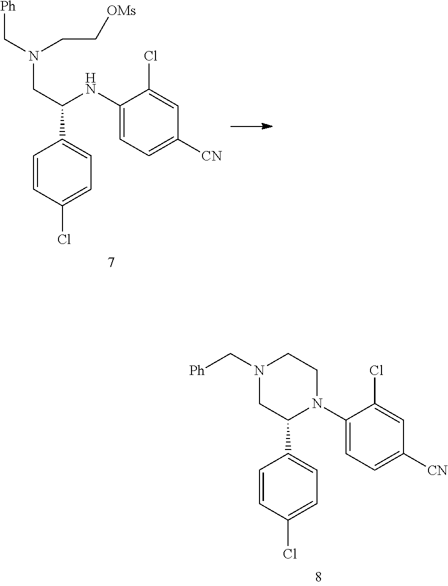 [SMILES: CS(=O)(=O)OCCN(Cc1ccccc1)C[C@H](Nc1ccc(C#N)cc1Cl)c1ccc(Cl)cc1 ; N#Cc1ccc(N2CCN(Cc3ccccc3)C[C@H]2c2ccc(Cl)cc2)c(Cl)c1]

cyclizing compound 7, wherein Ms is mesyl and Ph is phenyl, by treating with sodium hydroxide to obtain 8;

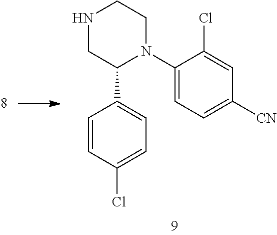 [SMILES: N#Cc1ccc(N2CCNC[C@H]2c2ccc(Cl)cc2)c(Cl)c1]

removing the benzyl protecting group from compound 8 to obtain 9; and

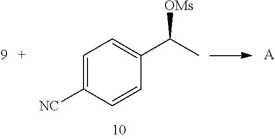

reacting compound 9 with compound 10, wherein Pr is mesyl, in the presence of potassium carbonate.

Following is a detailed description of the steps in the process of the invention. In the description, the intermediates are referred to as those in the process for preparing the compound of Formula A, but the process descriptions are applicable to the corresponding processes for preparing the compounds of formula I and II (e.g., the process for 8=the process for 8a=the process for 8b).

In Step a), approximately equimolar amounts of the protected amino ethanol 1 and 4-chloro-styreneoxide (2) are reacted to obtain compound 3. No solvent is required for the reaction. Compound 2 is added to heated compound 1 over about 4 to about 8 hours, preferably about 6 hours; reversal of the order of addition, i.e., adding 1 to 2, resulted in increased byproduct formation. The reaction is conducted at elevated temperatures of about 100 to about 150° C., preferably about 120 to about 130° C., more preferably about 125° C.; higher reaction temperatures increased formation of byproducts, while lower reaction temperatures required longer reaction times. Compound 3 is typically obtained in greater than 90% yield. The resultant product can be used without further purification in the next step.

In Step b), compound 3 is dissolved in a solvent such as 2-Me-THF, DCM, t-butylmethylether (TBME) or tetrahydrofuran (THF), with 2-Me-THF being preferred, and a base is added, preferably triethylamine (TEA), at a ratio of about 2-3 to 1 (base to 3), preferably about 2.3:1. The reaction mixture is cooled to about 0 to about 10° C., preferably about 0 to about 5° C., more preferably about 5° C., and a slight excess of methanesulfonyl chloride (MSCI) is added, followed by a slight excess of compound 4. The resultant mixture is heated to reflux, then cooled and filtered to obtain compound 5 in the filtrate, which is used in the next step without further purification.

In Step c), the filtrate containing compound 5 from Step b) is treated to cleave the protecting group, resulting in the formation of a salt, preferably the HCl, p-toluenesulfonic (p-TOS) acid or 1,5-naphthalenedisulfonic (napadisylate) salt. For the p-TOS salt, the filtrate is diluted with methanol, and p-toluenesulfonic acid is added in a molar ratio of about 1:1.5 (5 to acid) while maintaining a temperature of about 15 to about 25° C., preferably about 20° C. Seed crystals are preferably added to initiate precipitation of compound 6, and the precipitate is recovered under lowered temperatures (about 5 to about −15° C.) and dried at elevated temperatures to obtain compound 6.

The solvent switch to methanol provides a faster reaction and better precipitation/improved filterability compared to the previously reported method, and does not require temperatures below 0° C. The yield is improved by performing the aging and isolation of the precipitate at lower temperatures.

For the preparation of the HCl salt, the compound 5 is converted to the tosylate as described above, then water is added and the pH is adjusted to neutral by the addition of aqueous NaOH. The solids are filtered, re-slurried in 2-Me-THF, and filtered again. The combined organic layers are dried and concentrated by distillation. The residue is dissolved ina combination of 2-Me-THF and 2-propanol (about 3:2) and a slight molar excess of HCl in diethyl ether is added slowly at 20-23° C. The precipitated salt is filtered off, washed with 2-Me-THF and dried at 50-55° C.

In Step d), the mesyl protecting group is introduced by suspending compound 6 in a solvent such as 2-Me-THF or THF, preferably 2-Me-THF, and adding a base such as TEA in a ratio of about 2-3 to 1 (base to 6), preferably about 2.7:1. The reaction mixture is cooled to about 0 to about 10° C., preferably about 5° C., and a slight excess of MsCl is added while maintaining the temperature at about 5 to about 10° C. After warming to room temperature, an aqueous extraction is performed, and the separated organic layer containing compound 7 is used without further work-up in the next step.

The aqueous extraction preferably involves washing the reaction mixture with saturated aqueous NaHCO₃ solution, separating the organic layer, washing the organic layer with brine, and recovering the organic layer.

In Step e), ring closure is effected by refluxing the organic solution of compound 7 with NaOH in a ratio of about 2:1 (NaOH to compound 7). After cooling and aqueous extraction, the organic layer containing compound 8 can be evaporated to dryness, or preferably is used in the next step without further workup.

The aqueous extraction preferably comprises washing the room temperature organic solution with water, extracting the aqueous layer with 2-Me-THF, combining the organic layers and washing the organic layer with brine

In Step f), the benzyl protecting group is removed by methods known in the art, preferably by treating a solution of compound 8 in DCM or 2-Me-THF with an excess of 1-chloroethyl chloroformate at about 0 to about 5° C., then at about 20° C. The solvent is removed and the resultant residue is extracted to obtain compound 9 as an oil that tends to. crystallize with time.

A preferred extraction procedure is to perform a solvent switch from the DCM or 2-Me-THF to methanol, partitioning the resultant oil between water and TBME, basifying the aqueous layer, and extracting the aqueous layer with ethyl acetate; the ethyl acetate phase is washed with brine and concentrated.

De-benzylation using Pd/C under acidic conditions is an alternative procedure. In Step g), approximately equimolar amounts of compound 9 and compound 10 are refluxed with a base such as potassium carbonate in a solvent such as acetonitrile or 2-Me-THF. The preferred protecting group (Pr) is mesyl. The resultant solid is removed by filtration, the filtrate is concentrated, and compound I is recovered from the resultant oil by crystallization, typically from diethyl ether.

The compound of formula A can be further purified. A typical method involves dissolving the product in ethanol, concentrating, redissolving in a small amount of ethanol (to achieve a solvent switch) and adding TMBE. A slight excess of HCl (preferably 2 N in diethyl ether) is added and the precipitate is filtered and dried. The resultant salt is suspended in ethanol, heated to reflux, cooled and filtered. The salt is suspended in ethyl acetate and about 2 equivalents of aqueous potassium carbonate is added. The organic layer is separated, washed, dried and concentrated. The residue is dissolved in 2-propanol and seeded with crystals of compound A; the resulting precipitate is collected by filtration and dried.

The compounds of formula II and A are photosensitive, especially in solution, so the claimed process, in particular the final steps, should be conducted with minimal exposure to light.

The staring materials 1, 2 and 10 are known in the art, and can be prepared by known procedures, such as those described in U.S. Pat. No. 7,700,597.

### Specific Examples For the Preparation of Compounds II And A

### Preparation of Compound 3b

### Step a)

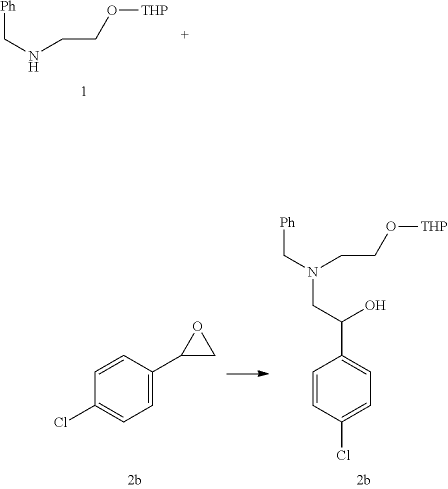

Compound 1 (311.0 g, 1.32 mol) was heated to 125° C. Compound 2b (204.7 g, 1.32 mol) was added at 125° C. (+/−3° C.) within 6 h above the surface. After a post reaction time of 2 h at 125° C. the yellow liquid was cooled to ambient temperature to give compound 3b (515.0 g, 1.32 mol, 92% yield, 85% LC purity).

### Preparation of Compound 5b

### Step b)

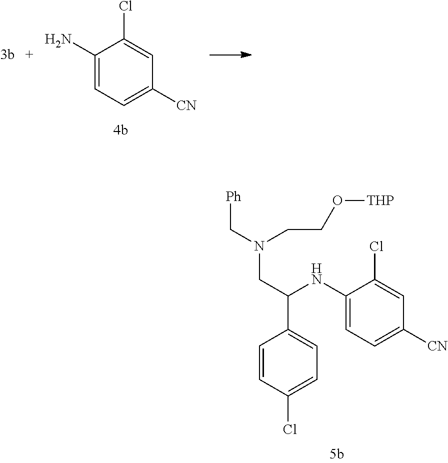

Compound 3b (390.0 g, 1.00 mol) was dissolved in 800 mL 2-Me-THF followed by the addition of triethylamine (TEA, 232.5 g, 2.30 mol). The light yellow solution was cooled to 5° C. Methanesulfonyl chloride (126.0 g, 1.10 mol) was added within 1 h while maintaining the temperature at 5° C. The reaction was continued for 1 h at 5° C. A solution of 4b (4-amino-3-chlorobenzonitrile) (167.0 g, 1.10 mol) in 300 mL 2-Me-THF was added and the slurry was heated to reflux (˜84° C.), followed by stirring under reflux for 4 h. The reaction mixture was cooled to 20° C. and the precipitate was removed by suction filtration. The product cake was washed with 200 mL 2-Me-THF. The mother liquor was used as such in the next step.

### Preparation of Compound 6b

### Step c)

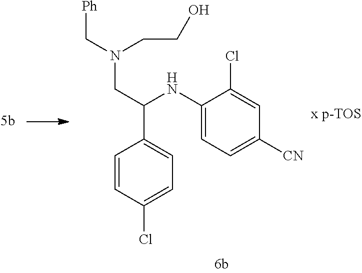 [SMILES: N#Cc1ccc(NC(CN(CCO)Cc2ccccc2)c2ccc(Cl)cc2)c(Cl)c1]

Compound 5b from Step b) in 2-Me-THF (˜1500 mL, 1.00 mol) was diluted with 300 mL methanol. A solution of p-toluenesulfonic acid (275.0 g, 1.45 mol) in 300 mL methanol was added within 0.5 h maintaining the temperature at 20° C. After a post reaction time of 2 h, seed crystals (5.0 g) from a previous batch were added to initiate the product precipitation which started immediately upon seed addition. The resulting slurry was cooled to −10° C. within 2 h. After aging for 0.5 h the precipitate was filtered and washed with 200 mL pre-cooled methanol. The solid was dried at 40° C. over 24 h to provide racemic compound 6b (495.0 g, 0.73 mol, 73% yield, 90% purity by LC).

### Preparation of Compound 7b

### Step d)

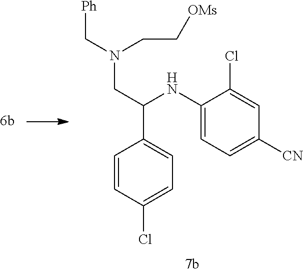 [SMILES: CS(=O)(=O)OCCN(Cc1ccccc1)CC(Nc1ccc(C#N)cc1Cl)c1ccc(Cl)cc1]

Racemic compound 6b (50.0 g, 0.08 mol) was suspended in 250 mL 2-Me-THF followed by the addition of TEA (23.1 g, 0.22 mol). The resulting solution was cooled to 5° C., followed by addition of methanesulfonyl chloride (15.0 g, 0.13 mol) within 0.5 h at 5-10° C. After stirring for 1 h at 5° C. the reaction mixture was allowed to warm to RT. 200 mL saturated sodium hydrogen carbonate solution was added and the mixture was stirred for 0.1 h. The phases were separated and the organic layer was washed with 50 mL brine. The resulting organic solution containing racemic compound 7b is used as such in the next step.

### Preparation of Compound 8b

### Step e)

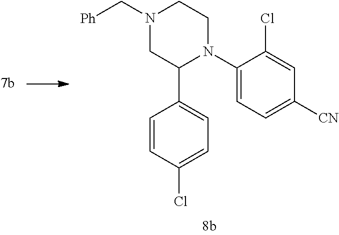 [SMILES: N#Cc1ccc(N2CCN(Cc3ccccc3)CC2c2ccc(Cl)cc2)c(Cl)c1]

To the organic solution (2-Me-THF) of racemic compound 7b (˜300 mL, 0.08 mol) NaOH (6.40 g, 0.16 mol) was added and the reaction mixture was stirred for 1 h under reflux. The reaction mixture was cooled to RT and 100 mL water were added. After stirring for 0.1 h, the phases were separated. The aqueous layer was extracted with 50 mL 2-Me-THF. The combined organic layers were washed with brine.

To determine the yield, concentration in vacuum yielded racemic compound 8b as yellow oil (29.0 g, 0.06 mol, 77% yield, 90% purity by LC).

### Preparation of Compound 9b

### Step f)

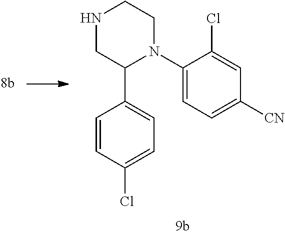 [SMILES: N#Cc1ccc(N2CCNCC2c2ccc(Cl)cc2)c(Cl)c1]

1-Chloroethyl chloroformate (12.3 g, 0.086 mol) was slowly added to a solution of racemic compound 8b (28.0 g, 0.066 mol) in 100 mL DCM at 0-5° C. within 0.5 h. The reaction mixture was stirred for 1 h at 5° C. and 1 h at 20° C. After concentration on rotary evaporator the residue was dissolved in 100 mL methanol and stirred for 1 h under reflux. The reaction mixture was again concentrated on rotary evaporator to complete the solvent switch. The remaining oil was stirred in 100 mL water and 100 mL TBME. The phases were separated. The aqueous layer was extracted with 50 mL TBME. After phase separation the aqueous layer was set to basic pH by adding 2N NaOH followed by two times extraction with 50 mL ethyl acetate. The combined organic phases were washed with brine and concentrated to give racemic compound 9b as an oil that tends to crystallize in time (24.0 g, 0.065 mol, 98% yield, 90% LC purity).

### Preparation of Compound II

### Step g)

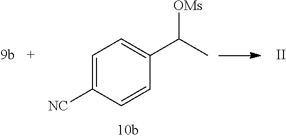

Racemic compound 9b (25.0 g, 0.075 mol), 10b (18.7 g, 0.083 mol) and potassium carbonate (31.1 g, 0.256 mol) were stirred under reflux for 10 h in 400 mL acetonitrile. The reaction mixture was cooled to RT followed by suction filtration of the solid. The filter cake was washed with 100 mL acetonitrile and the mother liquor was concentrated in vacuum. Crystallization of the remaining oil using 150 mL diethyl ether yielded compound I (22.2 g, 0.047 mol, 63% yield, 97% LC purity).

### Preparation of Compound 3

### Step a)

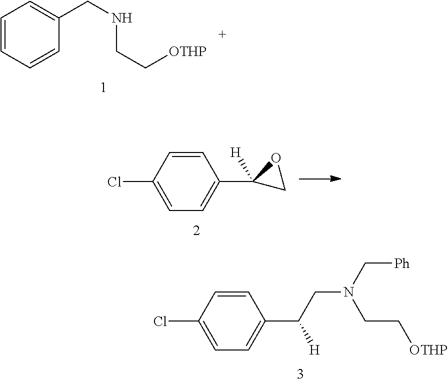

1 (701.5 g, 2.98 m) was charged to the flask and heated to 125° C. With stirring, 2 (4690.9 g, 2.98 m) was dripped in over 7 h and 47 min while maintaining the internal temperature in the range of 122.3-128.9° C. The reaction was stirred overnight at 125° C. for a total reaction time of about 24 h. The reaction was cooled to room temperature to provide 3 as a thick yellow syrup (1162 g).

### Preparation of 5

### Step b)

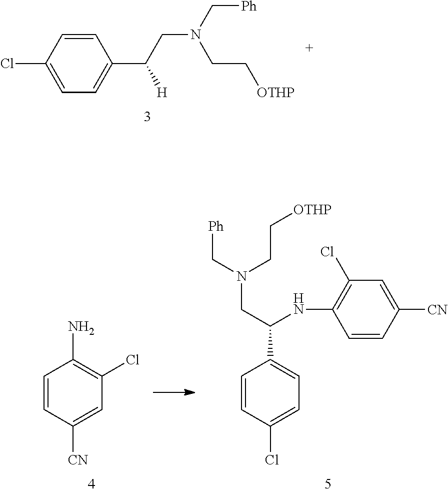

3 (1162 g, 2.98 m), 2-Me-THF (2.38 L) and TEA (694.1 g, 6.86 m) were charged to the flask and cooled to 0-5° C. Methanesulfonyl chloride (376.1 g, 3.28 m) was dripped in over 2.5 h while maintaining the internal temperature in the range of 0.8-4.7° C. After 1 h, 4 (501.5 g, 3.29 m) was added using 2-Me-THF (0.77 L) to wash it all in. The resulting suspension was heated to a gentle reflux and stirred overnight (about 18 h). The reaction mixture was cooled to 21° C. and filtered. The filter cake was suspended in 2-Me-THF (1.5 L) and filtered again, washing with 2-Me-THF (0.3 L). The combined filtrate containing crude 5 weighed 5948.6 g.

### Preparation of 6

### Step c)

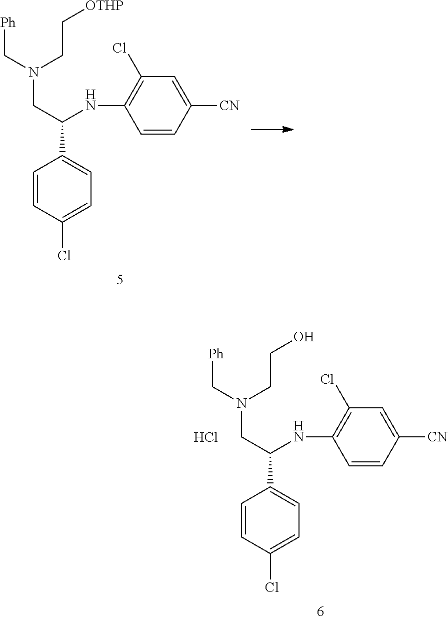

5 solution from the previous step (5948 g solution, assumed 1563 g of 5, 2.98 m) was charged to the flask. Methanol (0.89 L) was added and the mixture was cooled to 10° C. A solution of pTsOH monohydrate (822 g) in methanol (0.89 L) was added over 30 min. The internal temperature rose to 17.3° C. during the addition. The reaction was stirred at 20° C. for 4 h.

Water (0.89 L) was added, and the pH was adjusted to ˜11 using 20 wt % NaOH (1156.7 g in total). The solids which formed during neutralization were removed by filtration (slow). The wet cake was reslurried in 2-Me-THF (3 L) and filtered. Separately, the two filtrates were allowed to phase-separate (slow). The organic layers were combined and dried over sodium sulfate, which was removed by filtration with a 2-Me-THF wash (0.5 L). The filtrate still contained 4.5 wt % of water by KF analysis. The solution was concentrated and dried by distillation of 2-Me-THF (4 L). The solution was concentrated to a final weight of 2803 g, with a moisture content of 0.48 wt % water by KF titration.

The solution was diluted with 2-Me-THF (3.03 L) and 2-propanol (1.96 L). The 2N HCl in ether (1.636 L, 3.272 m) was added over about 45 min at 20-23° C. During the addition, the 6 HCl salt precipitated to form a thick suspension which was stirred for another 2 h at room temperature. The salt was collected on a vacuum filter and washed with 2-Me-THF (0.7 L). The salt was dried in a vacuum oven at 50-55° C. for 3.5 days. The yield of 6 was 1299.4 g of off-white powder. By ¹H NMR estimation, the product contained 11.7 wt % of 2-MeTHF, 0.6 wt % 2-PrOH, and 1.4 wt % of Et₃N-HCl salt. The corrected weight was 1121 g, a 79% overall yield for steps 1-3. The purity was estimated to be 98.1 AREA %.

### Preparation of 7

### Step d)

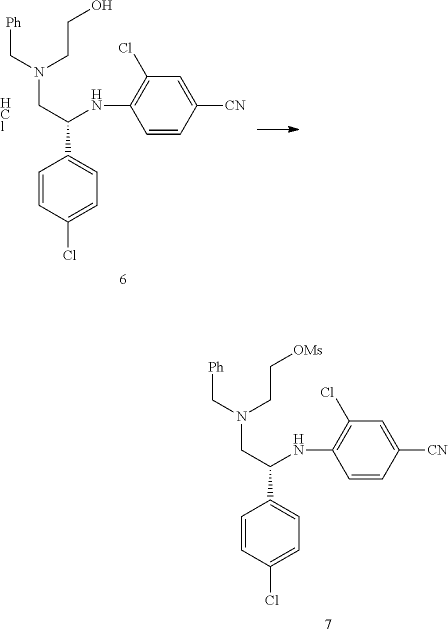 [SMILES: Cl ; N#Cc1ccc(N[C@@H](CN(CCO)Cc2ccccc2)c2ccc(Cl)cc2)c(Cl)c1 ; CS(=O)(=O)OCCN(Cc1ccccc1)C[C@H](Nc1ccc(C#N)cc1Cl)c1ccc(Cl)cc1]

6 HCl salt (650 g, 1.36 m) and 2-Me-THF (4.2 L) were charged to the flask. TEA (372.46 g, 3.68 m) was added and the suspension was cooled to 0-5° C. Methanesulfonyl chloride (187.4 g, 1.63 m) was added over 80 min. while maintaining the internal temperature at 9.2° C. The reaction was allowed to warm to about 12° C. over 1 h. The reaction was quenched with water (2.4 L). The organic layer was separated and washed with brine (0.75 L). The organic layer was held overnight in a freezer prior to Step e).

### Preparation of 8

### Step e)

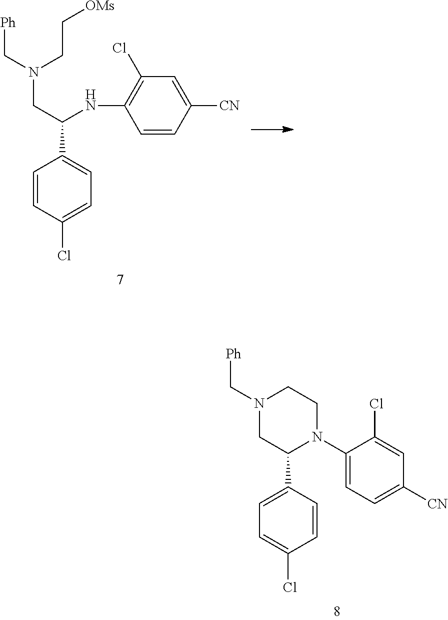 [SMILES: CS(=O)(=O)OCCN(Cc1ccccc1)C[C@H](Nc1ccc(C#N)cc1Cl)c1ccc(Cl)cc1 ; N#Cc1ccc(N2CCN(Cc3ccccc3)C[C@H]2c2ccc(Cl)cc2)c(Cl)c1]

The organic layer from Step d) was transferred to a 12-L flask and powdered NaOH (107.0 g, 2.67 m) was added. The suspension was heated to a gentle reflux (73-74° C.) for 1 h. The mixture was cooled and quenched with water (0.8 L). The organic layer was separated and washed with brine (0.8 L). The organic layer was concentrated under vacuum and dried by distillation of toluene (2×1.1 L). The yield was 585.6 g of viscous amber syrup. By ¹H NMR estimation, the syrup contained 15.5 wt % toluene and 1.4 wt % of isopropyl methanesulfonate ester. Corrected for solvent content of starting material and product, the yield was 97.9% for steps d) and e) overall. The purity of 8 was 95.6 AREA %, but the main peak was broad. About 0.5% of 6 was present.

### Preparation of 9

### Step f)

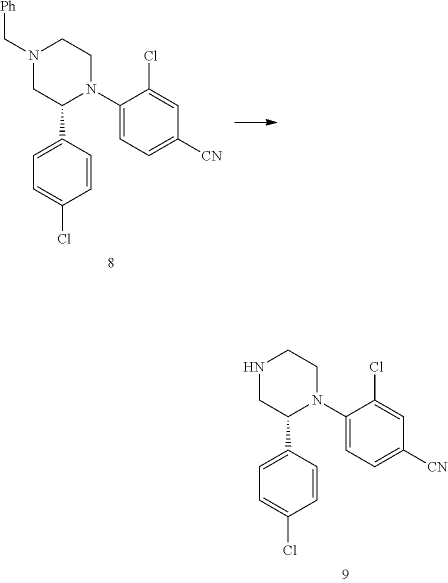 [SMILES: N#Cc1ccc(N2CCN(Cc3ccccc3)C[C@H]2c2ccc(Cl)cc2)c(Cl)c1 ; N#Cc1ccc(N2CCNC[C@H]2c2ccc(Cl)cc2)c(Cl)c1]

8 (581.5 g, 1.14 m) was dissolved in acetonitrile (2.1 L) and transferred to a 12-L flask. 1-chloroethyl chloroformate (206.7 g, 1.45 m) was added slowly over about 30 min at 20-24° C., then the reaction was stirred at room temperature for 2 h. Methanol (1.026 L) was added and the solution was heated and stirred at gentle reflux (60-63° C.) for 2 h. The reaction was cooled and concentrated under vacuum.

The residue was dissolved in water (4.2 L) and MTBE (2.1 L) with vigorous stirring. The layers were separated (slow, ˜200 ml of emulsion remained at the interface). The aqueous product layer (pH 1-2) was extracted with MTBE (2.1 L)—again slow separation, and some emulsion was present. The aqueous product layer was cooled to ˜10° C. Ethyl acetate (2.1 L) was added and the pH was adjusted to ˜12 using 20% aq. NaOH (462 g). The layers were separated and the aqueous layer was extracted with ethyl acetate (2.1 L). The combined organic product layers were dried over sodium sulfate (400 g), filtered and concentrated under vacuum to a residue, 400 g tan syrup. The yield corrected for solvents was about 91.4%.

### Preparation of A

### Step g)

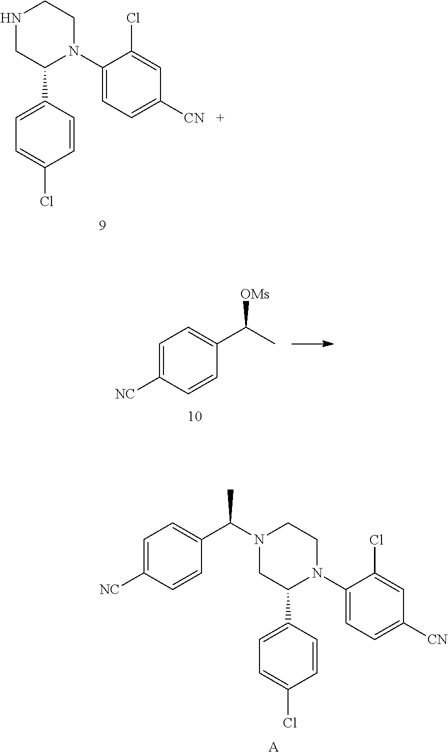

The residue of 9 (810 g, combined batches) was dissolved in acetonitrile (4 L) and transferred to a 12-L flask. 10 (507.2 g, 2.25 m), potassium carbonate powder (848.7 g, 6.14 m) and additional acetonitrile (2.1 L) were all added. The suspension was heated to gentle reflux (˜76-80° C.) and stirred overnight. After about 19 hours of reaction, the mixture was cooled and filtered to remove inorganic salts. The cake was washed with acetonitrile (2.5 L). The combined product filtrate was concentrated under vacuum to a residue, 1178 g.

### Purification of A, Form III

The crude residue of I was solvent exchanged by dissolution in ethanol (2×2 L) and concentration in the rotary evaporator. The residue was partly dissolved in ethanol (2.8 L total) in the rotary evaporator bulb with warming to ˜30° C. MTBE (1.8 L) was added, resulting in a clear amber solution. To the warm solution, 2 N HCl in diethyl ether (841 g, ˜1.126 L) was added while rotating the bulb. After about 10 min, the product quickly precipitated as a thick suspension which was rotated overnight in the bulb at ˜21° C. The product was collected on a vacuum filter and washed with cold 1:1 (v/v) ethanol/MTBE solution (1.2 L). After drying for 2 days in a vacuum oven at 45° C., the yield was 672.5 g of a light tan powder. The purity was 94.5 AREA %. By ¹H NMR estimation, the salt contained 0.24 wt % of MTBE and 0.94 wt % of ethanol.

The HCl salt (667 g) was transferred to a 12-L flask and suspended in ethanol (3.33 L). The suspension was heated to reflux (78.5° C.) for 30 min, then cooled naturally and stirred overnight at room temperature. The product was collected on a vacuum filter and washed with ethanol (0.7 L). After air-drying on the filter, the damp mass was 710 g. The purity was 98.7 AREA %. A sample of the filtrate was concentrated and dried—from 76.15 g filtrate was obtained 2.18 g solid, 2.86 wt5, therefore, about 75.7 g of solid was determined to be contained in the 2645 g of filtrate. The estimated yield was 591 g, 88.7%.

The damp HCl salt from reslurry was suspended in ethyl acetate (3 L). A 10% potassium carbonate solution (3 L) was added and stirred at room temperature for 20 min. until the solids dissolved. The layers were separated and the organic layer was washed with water twice (1 L each). The organic layer was dried over sodium sulfate, filtered, and concentrated. The residue was dissolved in ethyl acetate (2 L) and concentrated again to a residue.

The residue was mixed with 2-propanol (2 L, not well dissolved) and concentrated to a residue. The residue was dissolved in 2-propanol (6 L) in the rotary evaporator bulb with heating to 70° C., then about 3 L was removed under low vacuum to provide a clear solution (2353 g). Seeds of Form III (3 g) were added and rotated in the bath at 58-60° C. Very quickly a suspension formed, which was rotated at 58-60° C. overnight, a total of 11-12 h at that temperature. The suspension was allowed to cool to room temperature and rotate for 4 hours. The product was collected on a vacuum filter and washed with 2-propanol (1.5 L). The wet cake was dried on the filter under nitrogen for 1 day to provide 514 g powder containing about 0.8 wt % 2-propanol by ¹H NMR estimation. The powder was dried in a vacuum oven at 50° C. for 4 days. The 2-propanol level (estimated by NMR) leveled out at 0.44 wt %. The powder was passed through a 20 mesh s.s. screen to break up lumps. The final yield was 508.6 g (˜93% from the reslurried HCl salt).

### Preparation of Compound 1

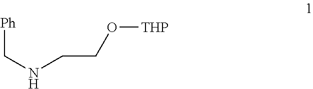

2-Benzylaminoethanol (500.0 g, 3.31 mol) was dissolved in 1.7 L DCM and cooled to 10° C. Methanesulfonic acid (349.5 g, 3.64 mol) was added within 45 min at 10-16° C. After 10 min, 3,4-dihydro-2H-pyran (472.9 g, 5.62 mol) was added within 30 min at 10-15° C. The solution was stirred for 1 h at 15° C. and was then added to a well stirred 3 N caustic solution (1.7 L, 4.91 mol) at 5-10° C. within 60 min. The phases were separated (2 min, 8° C.) and the organic layer was concentrated in vacuum without drying.

The oily yellow crude product (857 g) was purified by short path distillation at 3×10⁻³ mbar and 118-125° C. to give compound 1 (777.0 g, 3.30 mol, 99% yield, 96 area% LC purity).

### Preparation of Compound 2b

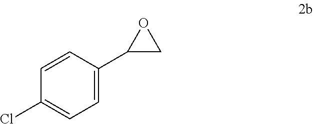

2-Bromo-4′-chloroacetophenone (580.0 g, 2.48 mol) was suspended in 5 L ethanol. Sodium borohydride (103.4 g, 2.73 mol) was added in 15 portions at 20-40° C. while cooling. The slurry was stirred over night at ambient temperature. Most of the ethanol was distilled off in vacuum and the remaining white solid mass was dissolved in 2.4 L water and 2.4 L DCM. The mixture was stirred for 20 min and the phases were separated. The aqueous layer was extracted with 1.0 L DCM and the combined organic layers were washed with 0.5 L water. The organic layer was dried with Na₂SO₄ and concentrated in vacuum.

The oily light yellow crude product was purified by short-path-distillation at 1×10⁻³ mbar and 60-65° C. to give racemic 2b (290.0 g, 1.73 mol, 70% yield, 92.6% quantitative NMR purity).

Compound 10b is prepared as described in U.S. Pat. No. 7,700,597.

### Preparation of 10

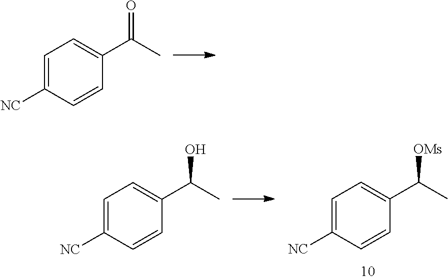

4-Acetylbenzonitrile (560.1 g, 3.858 m) was dissolved in THF (3.43 L) and cooled to about −20° C. (3aR)-Tetrahydro-1-methyl-3,3-diphenyl-1H,3H-pyffolo[1,2-c][1,3,2]oxaborazole (R)-Methyl-CBS) solution (548.7 g, 0.578 m, in toluene) was added over 7 min, while the internal temperature rose to −15.5° C. The solution was cooled to −20° C. and 2 M dimethylsulfide trihydroborane (BH₃-SMe₂) solution (1087.2 g, 2.543 m in THF) was dripped in over 2 h and 43 min at an internal temperature of ≦−12.8° C. After another 30 min, methanol (940 ml) was added over 33 min while the temperature rose to −1.5° C. During the addition, foaming and gas evolution occurred. The flask was vented through a calcium hypochlorite scrubber solution (about 400 g of Ca(OCl)₂ in 5-6 L of water). The reaction mixture was warmed to room temperature and sparged with nitrogen, venting through the scrubber solution for another 2.5 h to remove most of the dimethyl sulfide.

The reaction mixture was concentrated under vacuum to a yellow oil (917 g). The oil was dissolved in MTBE (2.2 L), poured into a solution of c. HCl (69.1 g) in water (1 L), and stirred for 23 min to precipitate out the hydrochloride salt of the chiral ligand. The ligand was removed by filtration (140 g after air-drying). The layers of the filtrate were separated (aq. pH=0-1). The organic layer was washed with water (1 L, pH˜3) and brine (1 L), and dried over sodium sulfate (175 g). The organic layer was concentrated to 558 g of oil. Since it appeared that some water may have come over in the distillate, the oil was dried by distilling 600 ml toluene, yielding 557 g of yellowish oil. By ¹H NMR analysis the oil contained 3 wt % toluene. The corrected yield was 540 g (95%). By chiral HPLC analysis, about 2.3% of enantiomer was present (95.4% e.e.).

The product was dissolved in DCM (2.22 L) and cooled to <5° C. TEA (354.9 g) was added. The solution was cooled to −2.6° C. and methanesulfonyl chloride (306.1 g) was added over 1 h 38 min while maintaining the internal temperature 7.1° C. An additional portion of methanesulfonyl chloride (30.2 g) was added at −1 to 4° C. After 20 min, the reaction was quenched with a mixture of 1 N HCl (600 ml) and water (1 L). The layers were separated (aq. pH=0-1) and the organic layer was washed with water (1.6 L) and brine (0.5 L). The organic layer was dried over sodium sulfate, filtered and concentrated under vacuum (bath=30° C. max) to a golden-colored oil, 557 g. By ¹H NMR analysis, the oil contained about 2.4 wt % 10 and 1.4 wt % of DCM. The corrected yield was 535 g (˜94%).

We claim:

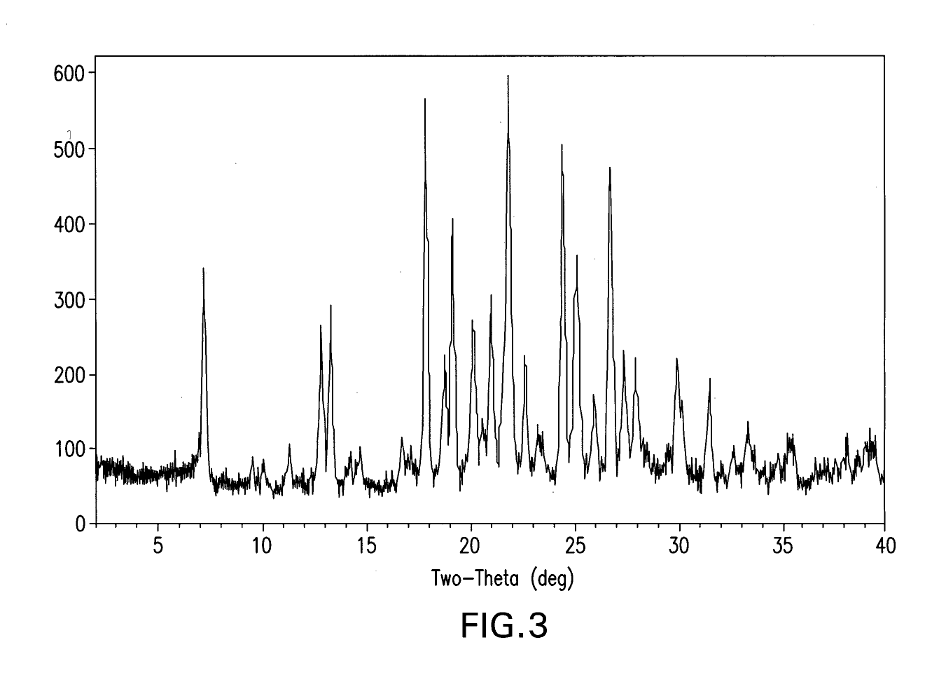

1. A crystalline polymorph of Compound A of the formula: 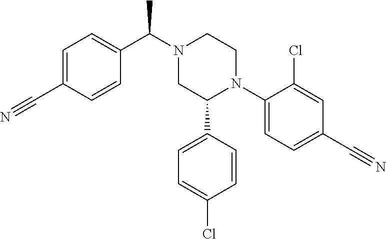
wherein, said polymorph is
Form 3 that exhibits a powder x-ray diffraction pattern substantially the same as the pattern shown in FIG. 3.

2-9. (canceled)

10. A crystalline polymorph Form III of Compound A of the formula: 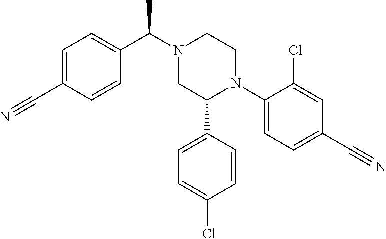 [SMILES: C[C@H](c1ccc(C#N)cc1)N1CCN(c2ccc(C#N)cc2Cl)[C@H](c2ccc(Cl)cc2)C1]
that exhibits a powder x-ray diffraction pattern having characteristic peak locations of 17.9, 21.9, 24.5 and 26.8 degrees 2θ.

11. The crystalline polymorph Form III of claim 10 that exhibits a powder x-ray diffraction pattern having characteristic peak locations of 7.3, 13.4, 17.9, 19.2, 21.9, 24.5, 25.2 and 26.8 degrees 2θ.

12. The crystalline polymorph Form III of claim 10 that exhibits a powder x-ray diffraction pattern having characteristic peak locations of 7.277, 12.898, 13′.398, 17.900, 19.238, 20.100, 21.058, 21.625, 21.939, 24.501, 25.179 and 26.800 degrees 2θ.

13-17. (canceled)

18. The crystalline polymorph Form III of Compound A that exhibits a melt substantially the same as shown in the differential scanning calorimetry scan of FIG. 6.

19. The crystalline polymorph Form III of claim 18 having a characteristic melt of 154.8° C.

20-21. (canceled)

22. A process for preparing the polymorph Form III of claim 1 from Compound A: 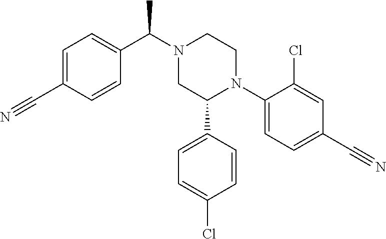 [SMILES: C[C@H](c1ccc(C#N)cc1)N1CCN(c2ccc(C#N)cc2Cl)[C@H](c2ccc(Cl)cc2)C1]
comprising the steps of:
a. mixing Compound A at elevated temperature with a first quantity of an organic solvent to form a mixture;
b. adding water portion-wise until precipitate is detected;
c. adding a second quantity of the organic solvent;
d. heating the mixture to about 70° C.; and
e. allowing the mixture to stand at room temperature until Form III crystals precipitate.

23. The process of claim 22 wherein the organic solvent is n-propanol.

24. The process of claim 22 wherein the ratio of the first quantity to the second quantity is about 2:1.

25. A pharmaceutical composition comprising a crystalline polymorph of Form III, of claim 1, and at least one pharmaceutically acceptable excipient or carrier.

26. A purified form of the polymorph of claim 1.

27. A method of treating obesity and obesity-related diseases in an animal in need of such treatment, comprising administering to said animal an effective amount of at least one polymorph of claim 1.

28. The method of claim 27 wherein the animal is a dog.

29. The method of claim 27 further comprising the administration of at least one additional anti-obesity agent.

30. A process for preparing the compound of formula II 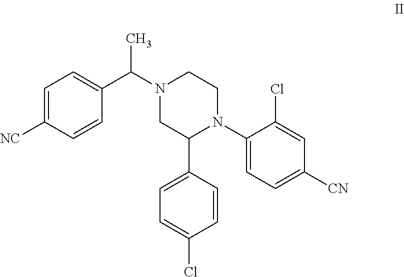 [SMILES: CC(c1ccc(C#N)cc1)N1CCN(c2ccc(C#N)cc2Cl)C(c2ccc(Cl)cc2)C1]
comprising: 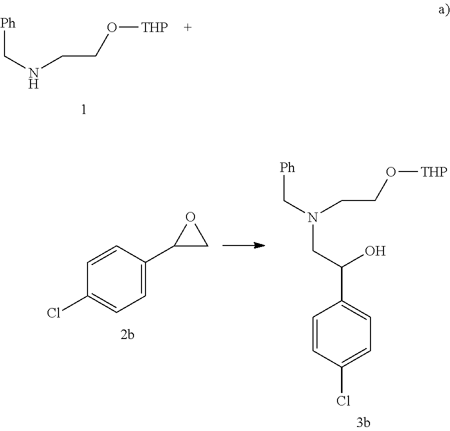
reacting the protected amino ethanol 1b and 4-chloro-styreneoxide (2b), wherein Ph is phenyl and THP is tetrahydropyranyl, to obtain compound 3b; 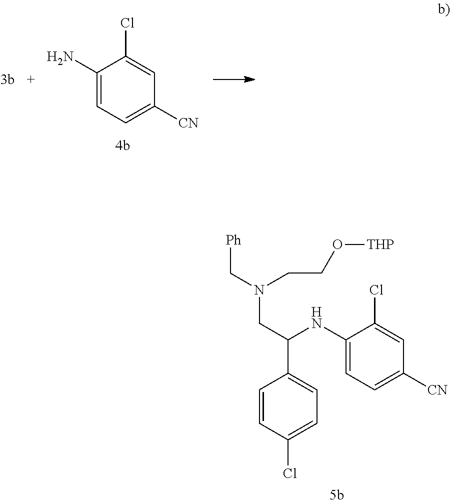
reacting compound 3b and 4-amino-3-chlorobenzonitrile (4b) in the presence of methanesulfonyl chloride and a base to obtain compound 5b; 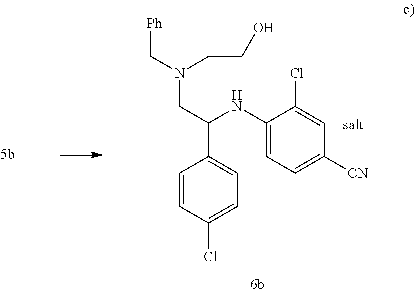 [SMILES: N#Cc1ccc(NC(CN(CCO)Cc2ccccc2)c2ccc(Cl)cc2)c(Cl)c1]
removing the THP protecting group from compound 5b and converting the product to a salt (6b); 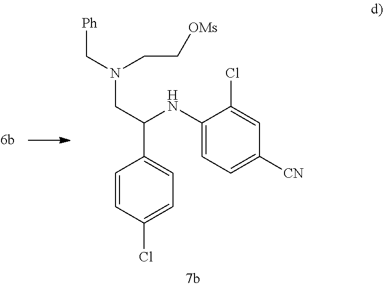 [SMILES: CS(=O)(=O)OCCN(Cc1ccccc1)CC(Nc1ccc(C#N)cc1Cl)c1ccc(Cl)cc1]
treating compound 6b with methanesulfonyl (Ms) chloride and a base to obtain compound 7b; 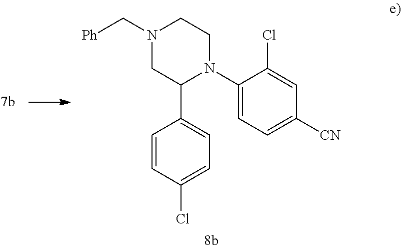 [SMILES: N#Cc1ccc(N2CCN(Cc3ccccc3)CC2c2ccc(Cl)cc2)c(Cl)c1]
cyclizing compound 7b by treating with sodium hydroxide to obtain 8b; 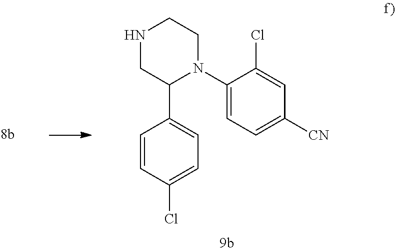 [SMILES: N#Cc1ccc(N2CCNCC2c2ccc(Cl)cc2)c(Cl)c1]
removing the benzyl protecting group from compound 8b; and 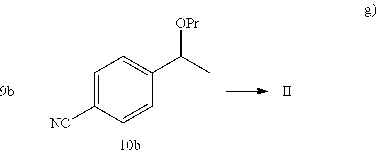
reacting compound 9b with compound 10b, wherein Pr is a protecting group, in the presence of potassium carbonate.

31. The process of claim 30 for preparing the compound of the Formula A 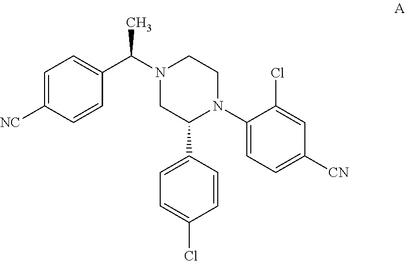 [SMILES: C[C@H](c1ccc(C#N)cc1)N1CCN(c2ccc(C#N)cc2Cl)[C@H](c2ccc(Cl)cc2)C1]
comprising: 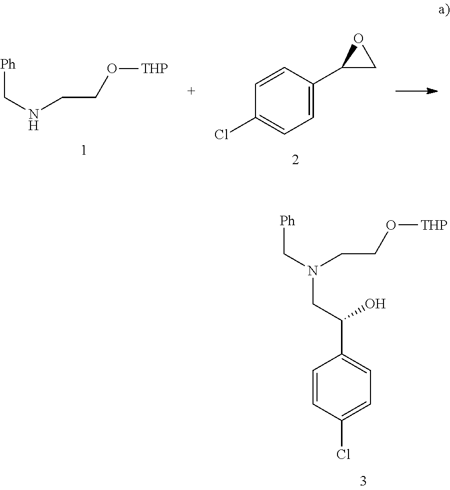
reacting the protected amino ethanol 1 and 4-chloro-styreneoxide (2), wherein Ph is phenyl and THP is tetrahydropyranyl, to obtain compound 3; 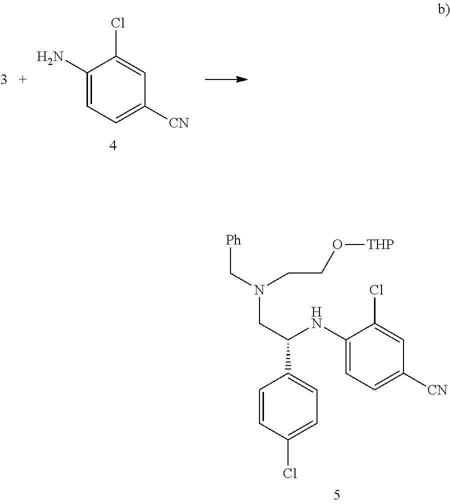
reacting compound 3 and 4-amino-3-chlorobenzonitrile (4) in the presence of methanesulfonyl chloride and a base to obtain compound 5; 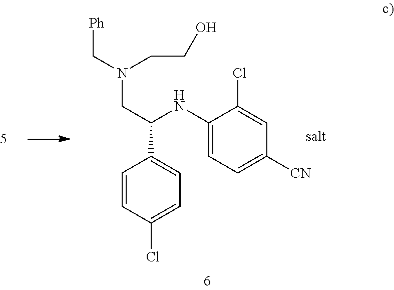 [SMILES: N#Cc1ccc(N[C@@H](CN(CCO)Cc2ccccc2)c2ccc(Cl)cc2)c(Cl)c1]
removing the THP protecting group from compound 5 and converting the product to a salt (6) 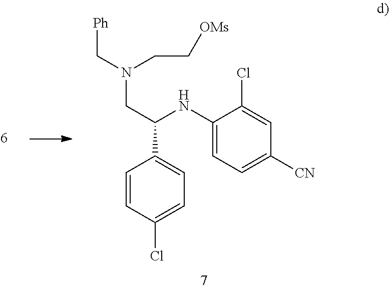 [SMILES: CS(=O)(=O)OCCN(Cc1ccccc1)C[C@H](Nc1ccc(C#N)cc1Cl)c1ccc(Cl)cc1]
treating compound 6 with methanesulfonyl (Ms) chloride and a base to obtain compound 7; 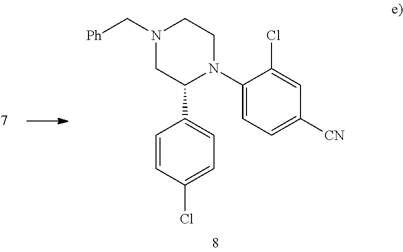 [SMILES: N#Cc1ccc(N2CCN(Cc3ccccc3)C[C@H]2c2ccc(Cl)cc2)c(Cl)c1]
cyclizing compound 7 by treating with sodium hydroxide; 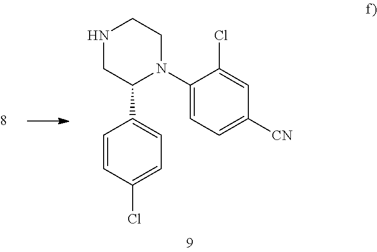 [SMILES: N#Cc1ccc(N2CCNC[C@H]2c2ccc(Cl)cc2)c(Cl)c1]
removing the benzyl protecting group from compound 8; and 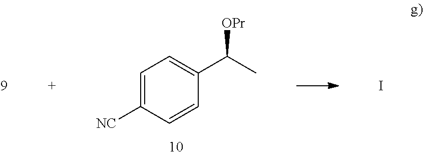
reacting compound 9 with compound 10, wherein Pr is a protecting group, in the presence of potassium carbonate.

32. The process of claim 31 comprising: 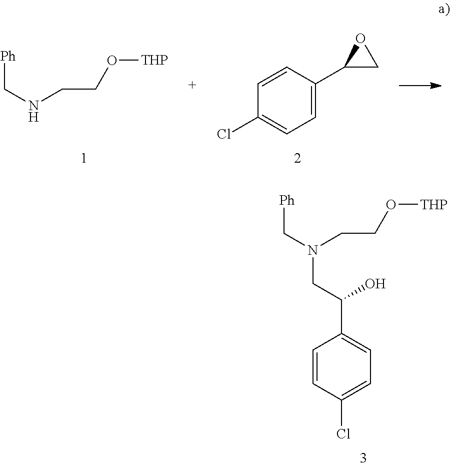
reacting the protected amino ethanol 1 and 4-chloro-styreneoxide (2) at about 125° C. to obtain compound 3; 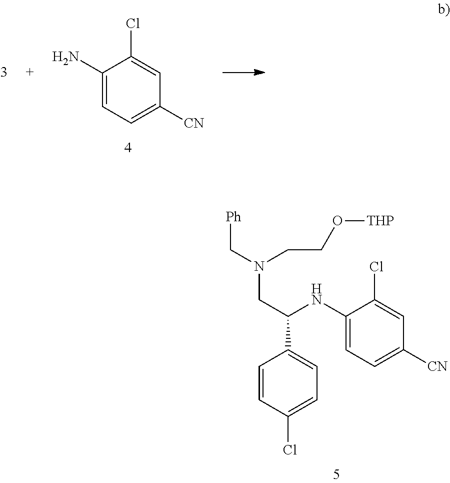
reacting compound 3 and 4-amino-3-chlorobenzonitrile (4) in 2-methyl-tetrahydrofuran the presence of methanesulfonyl chloride and triethylamine to obtain compound 5; 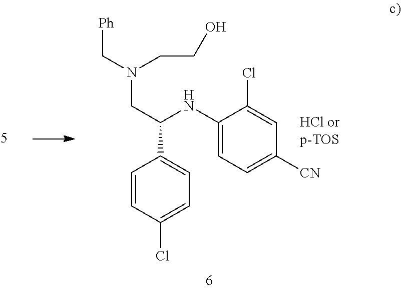 [SMILES: N#Cc1ccc(N[C@@H](CN(CCO)Cc2ccccc2)c2ccc(Cl)cc2)c(Cl)c1]
removing the THP protecting group from compound 5 and converting the product to the HCl or p-toluenesulfonic (p-TOS) salt (6) 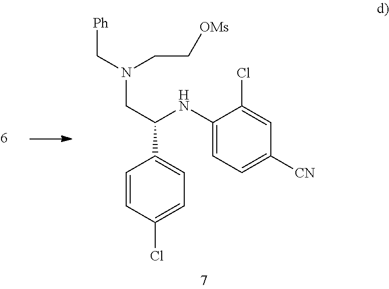 [SMILES: CS(=O)(=O)OCCN(Cc1ccccc1)C[C@H](Nc1ccc(C#N)cc1Cl)c1ccc(Cl)cc1]
treating compound 6 with methanesulfonyl (Ms) chloride and triethylamine to obtain compound 7; 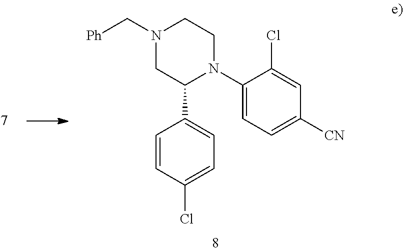 [SMILES: N#Cc1ccc(N2CCN(Cc3ccccc3)C[C@H]2c2ccc(Cl)cc2)c(Cl)c1]
cyclizing compound 7 by refluxing with sodium hydroxide; 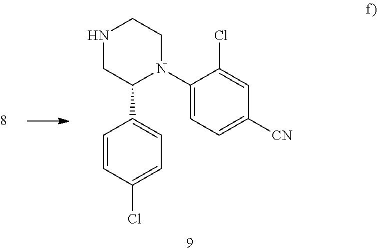 [SMILES: N#Cc1ccc(N2CCNC[C@H]2c2ccc(Cl)cc2)c(Cl)c1]
removing the benzyl protecting group from compound 8 by treating with 1-chloroethyl chloroformate; and 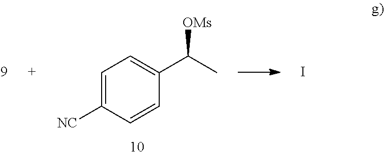
reacting compound 9 with compound 10 in the presence of potassium carbonate.

33. A process for preparing compound A 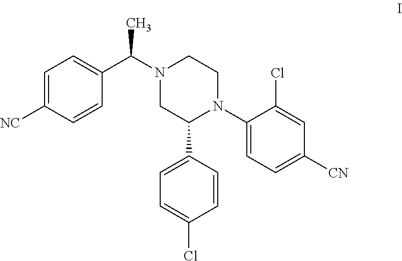 [SMILES: C[C@H](c1ccc(C#N)cc1)N1CCN(c2ccc(C#N)cc2Cl)[C@H](c2ccc(Cl)cc2)C1]
comprising: 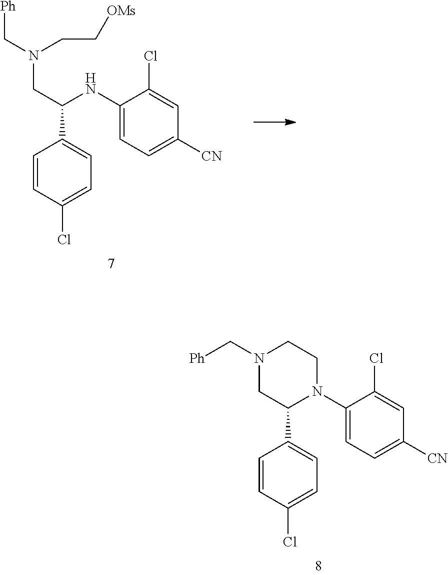 [SMILES: CS(=O)(=O)OCCN(Cc1ccccc1)C[C@H](Nc1ccc(C#N)cc1Cl)c1ccc(Cl)cc1 ; N#Cc1ccc(N2CCN(Cc3ccccc3)C[C@H]2c2ccc(Cl)cc2)c(Cl)c1]
cyclizing compound 7 by treating with sodium hydroxide to obtain compound 8; 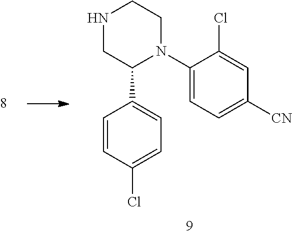 [SMILES: N#Cc1ccc(N2CCNC[C@H]2c2ccc(Cl)cc2)c(Cl)c1]
removing the benzyl protecting group from compound 8; and 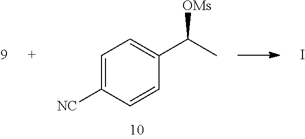
reacting compound 9 with compound 10 in the presence of potassium carbonate.

34. A compound selected from the group consisting of

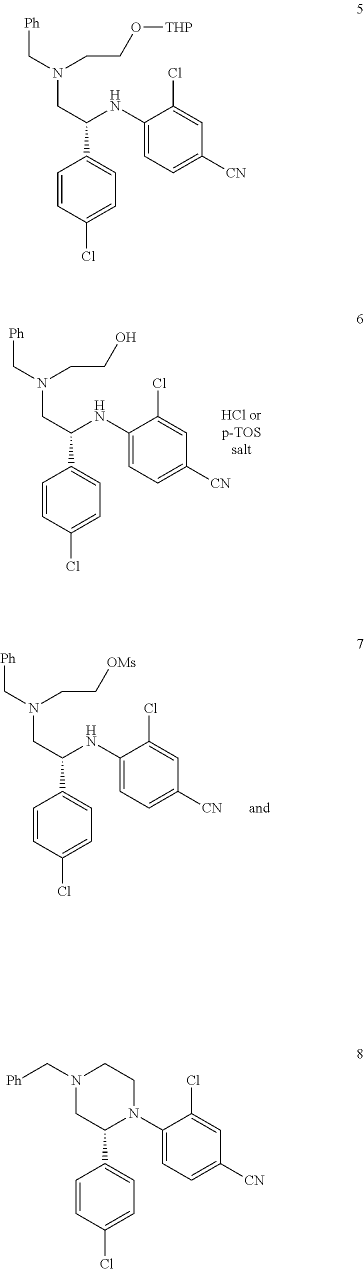

35. The compound selected from the group of claim 34, wherein the compound is 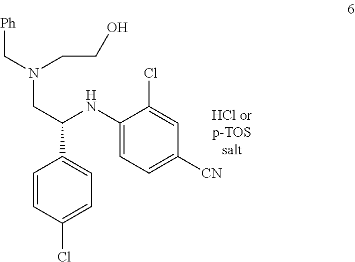 [SMILES: N#Cc1ccc(N[C@@H](CN(CCO)Cc2ccccc2)c2ccc(Cl)cc2)c(Cl)c1]
